(19) United States
(12) Patent Application Publication
(10) Pub. No.: US 2010/0098631 A1
(43) Pub. Date: Apr. 22, 2010

(54) Novel-2-Heteroaryl Substituted Indoles 695

(75) Inventors: Erwan Arzel, Tumba (SE); Britt-Marie Swahn, Sodertalje (SE); David Wensbo, Malmo (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 12/529,850

(22) PCT Filed: Mar. 5, 2008
(86) PCT No.: PCT/SE08/50242

§ 371 Date: Sep. 3, 2009

Related U.S. Application Data
(60) Provisional application No. 60/893,151, filed on Mar. 6, 2007.

(51) Int. Cl. A61K 51/00; C07D 209/04; C07D 401/04; C07D 403/04; A61K 31/4965; A61K 31/4439; A61K 31/405

(52) U.S. Cl. 424/1.65; 548/469; 546/277.4; 544/405; 514/255.05; 514/339; 514/415

(57) ABSTRACT

The present invention relates to novel 2-heteroaryl substituted indole derivatives, precursors thereof, and therapeutic uses of such compounds, having the structural formula (Ia) below: and to their pharmaceutically acceptable salt, compositions and methods of use. Furthermore, the invention relates to novel 2-heteroaryl substituted indole derivatives that are suitable for imaging amyloid deposits in living patients, their compositions, methods of use and processes to make such compounds. More specifically, the present invention relates to a method of imaging amyloid deposits in brain in vivo to allow antemortem diagnosis of Alzheimer's disease as well as measuring clinical efficacy of Alzheimer?s disease therapeutic agents.

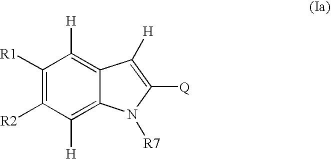

The present invention relates to novel 2-heteroaryl substituted indole derivatives and therapeutic uses for such compounds. Furthermore, the invention relates to novel 2-s heteroaryl substituted indole derivatives that are suitable for imaging amyloid deposits in living patients, their compositions, methods of use and processes to make such compounds. More specifically, the present invention relates to a method of imaging amyloid deposits in brain in vivo to allow antemortem diagnosis of Alzheimer's disease as well as measuring clinical efficacy of Alzheimer's disease therapeutic agents.

### BACKGROUND OF THE INVENTION

Amyloidosis is a progressive, incurable metabolic disease of unknown cause characterized by abnormal deposits of protein in one or more organs or body systems. Amyloid proteins are manufactured, for example, by malfunctioning bone marrow. Amyloidosis, which is occurs when accumulated amyloid deposits impair normal body function, can cause organ failure or death. It is a rare disease, occurring in about eight of every 1,000,000 people. It affects males and females equally and usually develops after the age of 40. At least 15 types of amyloidosis have been identified. Each one is associated with deposits of a different kind of protein.

The major forms of amyloidosis are primary systemic, secondary, and familial or hereditary amyloidosis. There is also another form of amyloidosis associated with Alzheimer's disease. Primary systemic amyloidosis usually develops between the ages of 50 and 60. With about 2,000 new cases diagnosed annually, primary systemic amyloidosis is the most common form of this disease in the United States. Also known as light-chain-related amyloidosis, it may also occur in association with multiple myeloma (bone marrow cancer). Secondary amyloidosis is a result of chronic infection or inflammatory disease. It is often associated with Familial Mediterranean fever (a bacterial infection characterized by chills, weakness, headache, and recurring fever), Granulomatous ileitis (inflammation of the small intestine), Hodgkin's disease, Leprosy, Osteomyelitis and Rheumatoid arthritis.

Familial or hereditary amyloidosis is the only inherited form of the disease. It occurs in members of most ethnic groups, and each family has a distinctive pattern of symptoms and organ involvement. Hereditary amyloidosis is though to be autosomal dominant, which means that only one copy of the defective gene is necessary to cause the disease. A child of a parent with familial amyloidosis has a 50-50 risk of developing the disease.

Amyloidosis can involve any organ or system in the body. The heart, kidneys, gastrointestinal system, and nervous system are affected most often. Other common sites of amyloid accumulation include the brain, joints, liver, spleen, pancreas, respiratory system, and skin.

Alzheimer's disease (AD) is the most common form of dementia, a neurologic disease characterized by loss of mental ability severe enough to interfere with normal activities of daily living, lasting at least six months, and not present from birth. AD usually occurs in old age, and is marked by a decline in cognitive functions such as remembering, reasoning, and planning.

Between two and four million Americans have AD; that number is expected to grow to as many as 14 million by the middle of the 21st century as the population as a whole ages. While a small number of people in their 40's and 50's develop the disease, AD predominantly affects the elderly. AD affects about 3% of all people between ages 65 and 74, about 20% of those between 75 and 84, and about 50% of those over 85. Slightly more women than men are affected with AD, even when considering women tend to live longer, and so there is a higher proportion of women in the most affected age groups.

The accumulation of amyloid AD-peptide in the brain is a pathological hallmark of all forms of AD. It is generally accepted that deposition of cerebral amyloid Aβ-peptide is the primary influence driving AD pathogenesis. (Hardy J and Selkoe D. J., Science. 297: 353-356, 2002).

Imaging techniques, such as positron emission tomography (PET) and single photon emission computed tomography (SPECT), are effective in monitoring the accumulation of amyloid deposits in the brain and correlating it to the progression of AD (Shoghi-Jadid et al. The American journal of geriatric psychiatry 2002, 10, 24; Miller, Science, 2006, 313, 1376; Coimbra et al. Curr. Top. Med. Chem. 2006, 6, 629; Nordberg, Lancet Neurol. 2004, 3, 519). The application of these techniques requires the development of radioligands that readily enter the brain and selectively bind to amyloid deposits in vivo.

A need exists for amyloid binding compounds that can cross the blood-brain barrier, and consequently, can be used in diagnostics. Furthermore, it is important to be able to monitor the efficacy of the treatment given to AD patients, by measuring the effect of said treatment by measuring changes of AD plaque level.

Properties of particular interest of a detectable amyloid-binding compound, besides high is affinity for amyloid deposits in vivo and high and rapid brain entrance, include low unspecific binding to normal tissue and rapid clearance from the same. These properties are commonly dependant on the lipophilicity of the compound (Coimbra et al. Curr. Top. Med. Chem. 2006, 6, 629). Among the proposed small molecules for imaging amyloid plaques, some uncharged analogs of thioflavin T of potential use have been synthesized (Mathis et al. J. Med. Chem. 2003, 46, 2740). Different isosteric heterocycles are reported as potential amyloid binding ligands (Cai et al. J. Med. Chem. 2004, 47, 2208; Ono et al. J. Med. Chem. 2006, 49, 2725; Jeong et al. Nucl. Med. Biol. 2006, 33, 811). Indole derivatives have previously been described for use in association with an amyloidogenic peptide (WO1995017095), and some indole derivatives have previously been described for use as amyloid imaging agents (WO04083195, WO2002085903). There is a need for improved compounds in order to obtain a signal-to-noise ratio high enough to allow detailed detection of amyloid deposits throughout all brain regions, and providing improved reliability in quantitative studies on amyloid plaque load in relation to drug treatments. The present invention provides novel 2-heteroaryl substituted indole derivatives for use as amyloid imaging agents and treatment of amyloid related diseases.

### DISCLOSURE OF THE INVENTION

In a first aspect of the invention, there is provided a compound according to formula Ia:

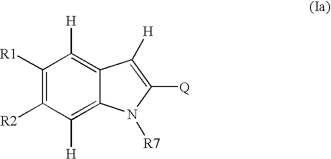

wherein
R1 is selected from H, halo, methyl, C₁₋₅ fluoroalkyl, C₁₋₃ alkyleneOC₁₋₃ alkyl, C₁₋₃ alkyleneOC₁₋₃ fluorolkyl, C₁₋₃ alkyleneNH₂, C₁₋₃ alkyleneNHC₁₋₃ alkyl, C₁₋₃ alkyleneN(C₁₋₃ alkyl)₂, C₁₋₃ alkyleneNHC₁₋₃ fluoroalkyl, C₁₋₃ alkyleneN(C₁₋₃ fluoroalkyl)₂, C₁₋₃ alkyleneN(C₁₋₃ alkyl)C₁₋₃ fluoroalkyl, hydroxy, methoxy, C₁₋₅ fluoroalkoxy, C₁₋₅ alkylthio, C₁₋₅ fluoroalkylthio, amino, NHC₁₋₃ alkyl, NHC₁₋₃ fluoroalkyl, N(C₁₋₃ alkyl)₂, N(C₁₋₃ alkyl)C₁₋₃ fluoroalkyl, NH(CO)C₁₋₃ alkyl, NH(CO)C₁₋₃ fluoroalkyl, NH(CO)C₁₋₃ alkoxy, NH(CO)C₁₋₃ fluoroalkoxy, NHSO₂C₁₋₃ alkyl, NHSO₂C₁₋₃ fluoroalkyl, (CO)C₁₋₃ alkyl, (CO)C₁₋₃ fluoroalkyl, (CO)C₁₋₃ alkoxy, (CO)C₁₋₃ fluoroalkoxy, (CO)NH₂, (CO)NHC₁₋₃ alkyl, (CO)NHC₁₋₃ fluoroalkyl, (CO)N(C₁₋₃ alkyl)₂, (CO)N(C₁₋₃ alkyl)C₁₋₃ fluoroalkyl, (CO)N(C₄₋₆ alkylene), (CO)N(C₄₋₆ fluoroalkylene), cyano, SO₂NHC₁₋₃ fluoroalkyl, nitro and SO₂NH₂;
R2 is selected from H, halo, methyl, C₁₋₅ fluoroalkyl, C₁₋₃ alkyleneOC₁₋₃ alkyl, C₁₋₃ alkyleneOC₁₋₃ fluorolkyl, C₁₋₃ alkyleneNH₂, C₁₋₃ alkyleneNHC₁₋₃ alkyl, C₁₋₃ alkyleneN(C₁₋₃ alkyl)₂, C₁₋₃ alkyleneNHC₁₋₃ fluoroalkyl, C₁₋₃ alkyleneN(C₁₋₃ fluoroalkyl)₂, C₁₋₃ alkyleneN(C₁₋₃ alkyl)C₁₋₃ fluoroalkyl, hydroxy, methoxy, C₁₋₅ fluoroalkoxy, C₁₋₅ alkylthio, C₁₋₅ fluoroalkylthio, amino, NHC₁₋₃ alkyl, NHC₁₋₃ fluoroalkyl, N(C₁₋₃ alkyl)₂, N(C₁₋₃ alkyl)C₁₋₃ fluoroalkyl, NH(CO)C₁₋₃ alkyl, NH(CO)C₁₋₃ fluoroalkyl, NH(CO)C₁₋₃ alkoxy, NH(CO)C₁₋₃ fluoroalkoxy, NHSO₂C₁₋₃ alkyl, NHSO₂C₁₋₃ fluoroalkyl, (CO)C₁₋₃ alkyl, (CO)C₁₋₃ fluoroalkyl, (CO)C₁₋₃ alkoxy, (CO)C₁₋₃ fluoroalkoxy, (CO)NH₂, (CO)NHC₁₋₃ alkyl, (CO)NHC₁₋₃ fluoroalkyl, (CO)N(C₁₋₃ alkyl)₂, (CO)N(C₁₋₃ alkyl)C₁₋₃ fluoroalkyl, (CO)N(C₄₋₆ alkylene), (CO)N(C₄₋₆ fluoroalkylene), cyano, SO₂NHC₁₋₃ fluoroalkyl, nitro and SO₂NH₂; or
R1 and R2 together forms a ring;

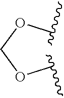

Q is a nitrogen-containing aromatic heterocycle selected from Q2 to Q10;

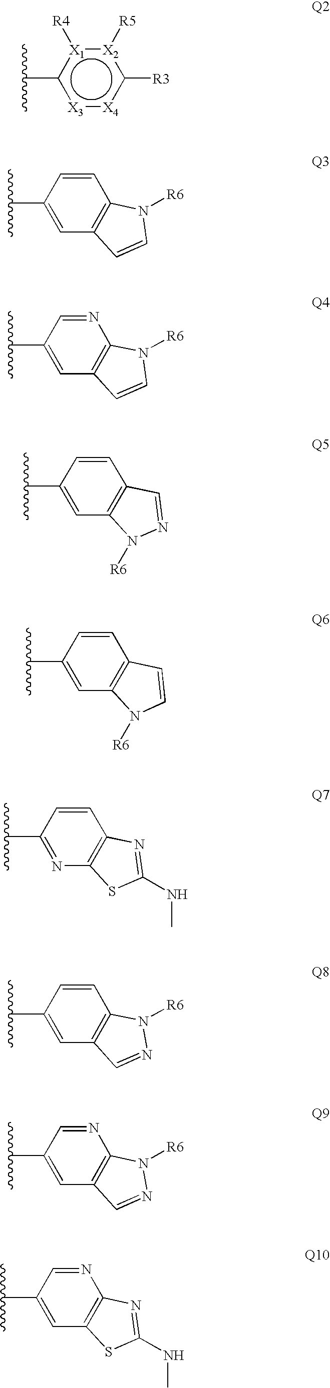

wherein
Q2 is a 6-membered aromatic heterocycle containing one or two N atoms, wherein X₁, X₂, X₃ and X₄ are independently selected from N or C; and wherein one or two of X₁, X₂, X₃ and X₄ is N and the remaining is C and when said X₁ is C, said C is optionally substituted with R4; and when said X₂ is C, said C is optionally substituted with R5;
R3 is selected from methoxy, C₁₋₄ fluoroalkoxy, amino, NHC₁₋₃ alkyl, NHC₁₋₃ fluoroalkyl, N(C₁₋₃ alkyl)₂, N(C₁₋₃ alkyl)C₁₋₃ fluoroalkyl, NH(CO)C₁₋₃ alkyl, NH(CO)C₁₋₃ fluoroalkyl, NH(CO)G2, (CO)NH₂, (CO)C₁₋₃ alkoxy, methylthio, C₁₋₆ fluoroalkylthio, SO₂NH₂, N(C₄₋₆ alkylene) and G1;

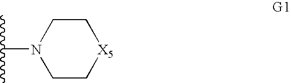

X₅ is selected from O, NH, NC₁₋₃ alkyl and N(CO)Ot-butyl;
G2 is phenyl, optionally substituted with a substituent selected from fluoro and iodo;
R4 is selected from H and halo;
R5 is selected from H, fluoro, bromo and iodo;
R6 is selected from H, methyl and (CH₂)₀₋₄CH₂F;
R7 is selected from H, methyl, (CO)C₁₋₄alkoxy and (CH₂)₀₋₄CH₂F;
wherein one or more of the constituting atoms optionally is a detectable isotope;
as a free base or a pharmaceutically acceptable salt, solvate or solvate of a salt thereof;
with the provisio that the following compounds are excluded:

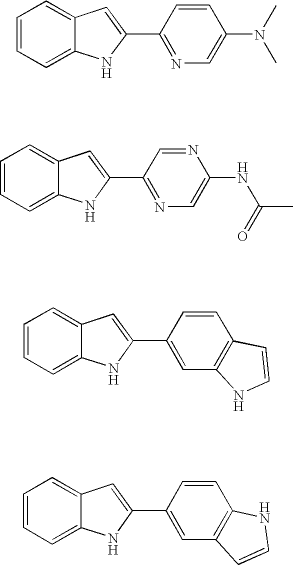

In another aspect of the invention, there is provided a compound according to formula Ia, wherein R4 is selected from H, fluoro, bromo and iodo.

In another aspect of the invention, there is provided a compound according to formula Ia, wherein R1 is selected from H, halo, methyl, C₁₋₅ fluoroalkyl, hydroxy, methoxy, C₁₋₅ fluoroalkoxy, methylthio, C₁₋₅ fluoroalkylthio, amino, NHmethyl, NHC₁₋₃ fluoroalkyl, N(CH₃)CH₃, N(C₁₋₃ alkyl)C₁₋₃ fluoroalkyl, NH(CO)C₁₋₃ alkyl, NH(CO)C₁₋₃ fluoroalkyl, NH(CO)C₁₋₃ alkoxy, NH(CO)C₁₋₃ fluoroalkoxy, NHSO₂C₁₋₃ alkyl, NHSO₂C₁₋₃ fluoroalkyl, (CO)C₁₋₃ fluoroalkyl, (CO)C₁₋₃ alkoxy, (CO)C₁₋₃ fluoroalkoxy, (CO)NH₂, (CO)NHC₁₋₃ fluoroalkyl, cyano, SO₂NHC₁₋₃ fluoroalkyl, nitro and SO₂NH₂; or

R1 and R2 together forms a ring;

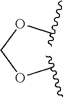

In another aspect of the invention, there is provided a compound according to formula Ia, wherein R1 is selected from H, fluoro, iodo, methyl, C₁₋₅ fluoroalkyl, hydroxy, methoxy, cyano, C₁₋₅ fluoroalkoxy, methylthio, amino, NHmethyl, NHC₁₋₃ fluoroalkyl, NH(CO)C₁₋₃ alkyl, NH(CO)C₁₋₃ fluoroalkyl, NH(CO)C₁₋₃ fluoroalkoxy, (CO)C₁₋₃ alkoxy and (CO)NH₂.

In another aspect of the invention, there is provided a compound according to formula Ia, wherein R1 is selected from H, fluoro, hydroxy and methoxy.

In another aspect of the invention, there is provided a compound according to formula Ia, is wherein R2 is selected from H, fluoro, iodo, C₁₋₅ fluoroalkyl, hydroxy, methoxy, (CO)NH₂, cyano and methylthio.

In another aspect of the invention, there is provided a compound according to formula Ia, wherein R2 is selected from H, fluoro, hydroxy and methoxy.

In another aspect of the invention, there is provided a compound according to formula Ia, wherein R2 is H.

In another aspect of the invention, there is provided a compound according to formula Ia, wherein Q is Q2.

In another aspect of the invention, there is provided a compound according to formula Ia, wherein Q is selected from Q3 to Q10.

In another aspect of the invention, there is provided a compound according to formula Ia, wherein Q2 is a pyridine ring, wherein X₃ and X₄ are independently selected from N or C, and wherein one of X₃ and X₄ is N and the remaining of X₁, X₂, X₃ and X₄ are C.

In another aspect of the invention, there is provided a compound according to formula Ia, wherein Q2 is a pyrimidine ring, wherein X₂ and X₄ are N, and wherein X₁ and X₃ are C.

In another aspect of the invention, there is provided a compound according to formula Ia, wherein Q2 is a pyrimidine ring, wherein X₁ and X₃ are N, and wherein X₂ and X₄ are C.

In another aspect of the invention, there is provided a compound according to formula Ia, wherein Q2 is a pyridazine ring, wherein X₃ and X₄ are N, and wherein X₁ and X₂ are C.

In another aspect of the invention, there is provided a compound according to formula Ia, wherein Q2 is a pyrazine ring, wherein X₁ and X₄ are N, and wherein X₂ and X₃ are C; or wherein X₁ and X₄ are C, and wherein X₂ and X₃ are N.

In another aspect of the invention, there is provided a compound according to formula Ia, wherein R3 is selected from methoxy, C₁₋₄ fluoroalkoxy, amino, NHC₁₋₃ alkyl, NHC₁₋₃ fluoroalkyl, N(C₁₋₃ alkyl)₂, N(C₁₋₃ alkyl)C₁₋₃ fluoroalkyl, NH(CO)C₁₋₃ alkyl, NH(CO)C₁₋₃ fluoroalkyl, (CO)NH₂, (CO)C₁₋₃ alkoxy, methylthio, C₁₋₆ fluoroalkylthio, SO₂NH₂, and G1; wherein X₅ is selected from O, NH and Nmethyl.

In another aspect of the invention, there is provided a compound according to formula Ia, wherein R3 is selected from NHmethyl, (CO)NH₂, (CO)methoxy.

In another aspect of the invention, there is provided a compound according to formula Ia, wherein R4 is selected from H and fluoro.

In another aspect of the invention, there is provided a compound according to formula Ia, wherein R4 is H.

In another aspect of the invention, there is provided a compound according to formula Ia, R5 is selected from H and fluoro.

In another aspect of the invention, there is provided a compound according to formula Ia, wherein R5 is H.

In another aspect of the invention, there is provided a compound according to formula Ia, wherein R6 is selected from H and methyl.

In another aspect of the invention, there is provided a compound according to formula Ia, wherein R6 is H.

In another aspect of the invention, there is provided a compound according to formula Ia, wherein R7 is selected from H, methyl and (CO)C₁₋₄alkoxy.

In another aspect of the invention, there is provided a compound according to formula Ia, wherein R7 is H or (CO)C₁₋₄alkoxy.

In another aspect of the invention, there is provided a compound according to formula Ia, said compound being:

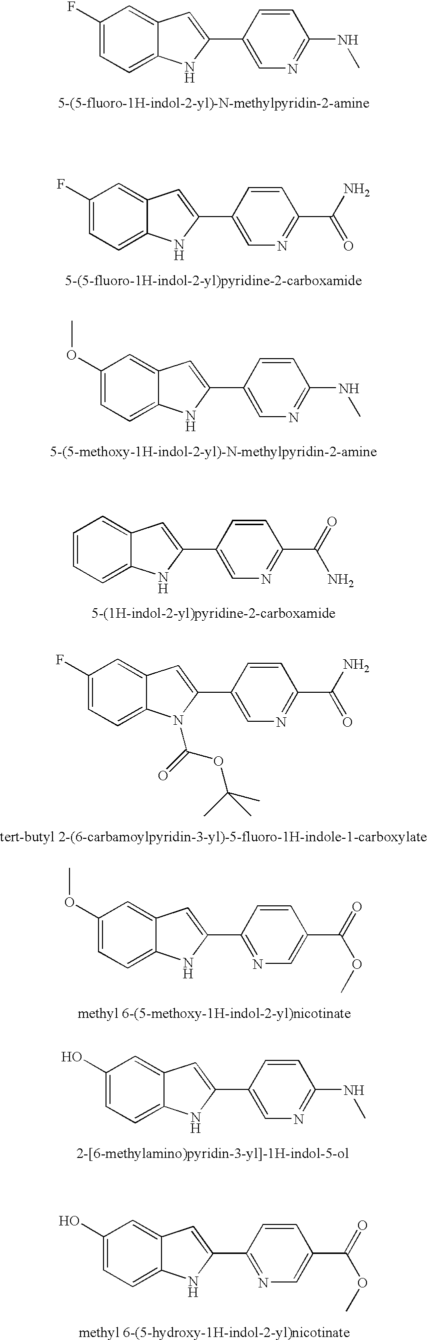

In another aspect of the invention, there is provided a compound, wherein one to six of the composing atoms is the detectable isotope ³H, or wherein one to three of the composing atoms is the detectable isotope ¹³C, or wherein one of the composing atoms is a detectable isotope selected from ¹⁸F, ¹¹C, ⁷⁵Br, ⁷⁶Br, ¹²⁰I, ¹²³I, ¹²⁵I, ¹³¹I and ¹⁴C, said compound being selected from:

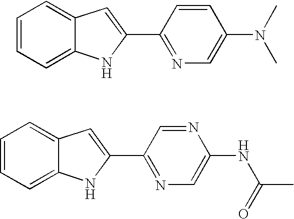

In one embodiment of this aspect, there is provided such a compound wherein one of the is composing atoms is the detectable isotope ¹¹C.

In another aspect of the invention, there is provided a compound according to formula Ia, wherein one or more of the atoms of the molecule represents a detectable isotope.

In another aspect of the invention, there is provided a compound according to formula Ia, wherein one to six of the composing atoms is the detectable isotope ³H, or wherein one to three of the composing atoms is a detectable isotope selected from ¹⁹F and ¹³C, or wherein one of the composing atoms is a detectable isotope selected from ¹⁸F, ¹¹C, ⁷⁵Br, ⁷⁶Br, ¹²⁰I, ¹²³I, ¹²⁵I, ¹³¹I and ¹⁴C.

In another aspect of the invention, there is provided a compound according to formula Ia, wherein one to six of the composing atoms is the detectable isotope ³H, or wherein one to three of the composing atoms is the detectable isotope ¹⁹F, or wherein one of the composing atoms is a detectable isotope selected from ¹⁸F, and ¹²³I.

In another aspect of the invention, there is provided a compound according to formula Ia, wherein one to six of the composing atoms is the detectable isotope ³H, or wherein one to three of the composing atoms is the detectable isotope ¹⁹F, or wherein one of the composing atoms is a detectable isotope selected from ¹⁸F and ¹¹C.

In another aspect of the invention, there is provided a compound according to formula Ia, wherein one of the composing atoms is the detectable isotope ¹¹C.

In another aspect of the invention, there is provided a compound according to formula Ia, wherein one of the composing atoms is the detectable isotope ¹⁸F.

In another aspect of the invention, there is provided a compound according to formula Ib:

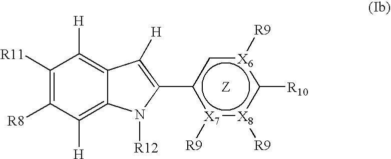

wherein
Z is a 6-membered aromatic heterocycle containing one or two N atoms, wherein X₆, X₇ and X₈ are independently selected from N or C, and wherein one or two of X₆, X₇ and X₈ is N and the remaining is C, and wherein X₆ is C, said C is optionally substituted with R9;
R8 is selected from OSi(G3)₃, OCH₂G4, OG5, H, bromo, fluoro, hydroxy, methoxy, Sn(C₁₋₄alkyl)₃, N(CH₃)₃⁺, IG6⁺, N₂⁺ and nitro;
R9 is selected from H, bromo, fluoro, Sn(C₁₋₄ alkyl)₃, N(CH₃)₃⁺, IG6⁺, N₂⁺ and nitro;
R10 is selected from amino, methylamino, dimethylamino, methoxy, hydroxy, (CO)NH₂, O(CH₂)₂₋₄G7 and NH(CH₂)₂₋₄G7;
R11 is selected from OSi(G3)₃, OCH₂G4, OG5, H, bromo, fluoro, hydroxy, methoxy, Sn(C₁₋₄ alkyl)₃, N(CH₃)₃⁺, IG6⁺, N₂⁺ and nitro;
R12 is selected from H, methyl, SO₂N(CH₃)₂, SO₂phenyl, SO₂(p-methyl)phenyl, CO₂CH₂CCl₃, CO₂(CH₂)₂Si(CH₃)₂, CO₂t-butyl, Si(G3)₃, P(═S)phenyl₂, and (CH₂)₂₋₄G7;
G3 is selected from C₁₋₄ alkyl and phenyl;
G4 is selected from 2-(trimethylsilyl)ethoxy, C₁₋₃ alkoxy, 2-(C₁₋₃ alkoxy)ethoxy, C₁₋₃ alkylthio, cyclopropyl, vinyl, phenyl, p-methoxyphenyl, o-nitrophenyl, and 9-anthryl;
G5 is selected from tetrahydropyranyl, 1-ethoxyethyl, phenacyl, 4-bromophenacyl, cyclohexyl, t-butyl, t-butoxycarbonyl, 2,2,2-trichloroethylcarbonyl and triphenylmethyl;
IG6⁺ represents a constituent of a iodonium salt, in which the iodo atom is hyper-valent and has a positive formal charge and, in which G6 is phenyl, optionally substituted with one substituent selected from methyl and bromo;
G7 is selected from bromo, iodo, OSO₂CF₃, OSO₂CH₃ and OSO₂phenyl, said phenyl being optionally substituted with methyl or bromo;
with reference to formula Ib, one or both of the following conditions are fulfilled: (1) R12 is H; (2) one or several of the substituents selected from R8, R9, R10, R11 is one of the functional groups selected from bromo, fluoro, hydroxy, Sn(C₁₋₄ alkyl)₃, N(CH₃)₃⁺, IG6⁺, N₂⁺, nitro, amino, methylamino, NH(CH₂)₂₋₄G7, O(CH₂)₂₋₄G7 and (CH₂)₂₋₄G7;
as a free base or a salt, solvate or solvate of a salt thereof;
with the provisio following compounds are excluded:

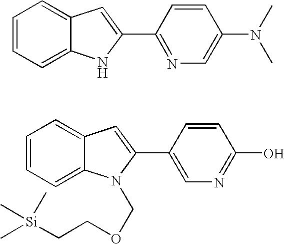

In another aspect of the invention, there is provided a compound according to formula Ib:

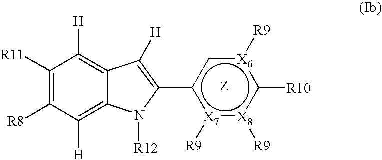

wherein
Z is a 6-membered aromatic heterocycle containing one or two N atoms, wherein X₆, X₇ and X₈ are independently selected from N or C, and wherein one or two of X₆, X₇ and X₈ is N and the remaining is C, and wherein X₆ is C, said C is optionally substituted with R9, and wherein X₇ is C, said C is optionally substituted with R9, and wherein X₈ is C, said C is optionally substituted with R9;
R8 is selected from OSi(G3)₃, OCH₂G4, OG5, H, bromo, fluoro, hydroxy, methoxy, Sn(C₁₋₄ alkyl)₃, N(CH₃)₃⁺, IG6⁺, N₂⁺ and nitro;
R9 is selected from H, bromo, chloro, iodo, fluoro, Sn(C₁₋₄alkyl)₃, N(CH₃)₃⁺, IG6⁺, N₂⁺ and nitro;
R10 is selected from amino, methylamino, dimethylamino, N(CH₃)CHO, N(CH₃)COCH₃, N(CH₃)CO₂-t-butyl, methoxy, hydroxy, (CO)NH₂, O(CH₂)₂₋₄G7 and NH(CH₂)₂₋₄G7;
R11 is selected from OSi(G3)₃, OCH₂G4, OG5, H, bromo, fluoro, hydroxy, methoxy, Sn(C₁₋₄ alkyl)₃, N(CH₃)₃⁺, IG6⁺, N₂⁺ and nitro;
R12 is selected from H, methyl, SO₂N(CH₃)₂, SO₂phenyl, SO₂(p-methyl)phenyl, CO₂CH₂CCl₃, CO₂(CH₂)₂Si(CH₃)₂, CO₂t-butyl, Si(G3)₃, P(═S)phenyl₂, and (CH₂)₂₋₄G7;
G3 is selected from C₁₋₄ alkyl and phenyl;
G4 is selected from 2-(trimethylsilyl)ethoxy, C₁₋₃ alkoxy, 2-(C₁₋₃ alkoxy)ethoxy, C₁₋₃ alkylthio, cyclopropyl, vinyl, phenyl, p-methoxyphenyl, o-nitrophenyl, and 9-anthryl;
G5 is selected from tetrahydropyranyl, 1-ethoxyethyl, phenacyl, 4-bromophenacyl, cyclohexyl, t-butyl, t-butoxycarbonyl, 2,2,2-trichloroethylcarbonyl and triphenylmethyl;
IG6⁺ represents a constituent of a iodonium salt, in which the iodo atom is hyper-valent and has a positive formal charge and, in which G6 is phenyl, optionally substituted with one substituent selected from methyl and bromo;
G7 is selected from bromo, iodo, OSO₂CF₃, OSO₂CH₃ and OSO₂phenyl, said phenyl being optionally substituted with methyl or bromo;
with reference to formula Ib, one or both of the following conditions are fulfilled: (1) R12 is H; (2) one or several of the substituents selected from R8, R9, R10, R11 is one of the functional groups selected from bromo, fluoro, hydroxy, Sn(C₁₋₄ alkyl)₃, N(CH₃)₃⁺, IG6⁺, N₂⁺, nitro, amino, methylamino, NH(CH₂)₂₋₄G7, N(CH₃)CHO, N(CH₃)COCH₃, N(CH₃)CO₂-t-butyl, O(CH₂)₂₋₄G7, OSi(G3)₃, OCH₂G4 and (CH₂)₂₋₄G7;
as a free base or a salt, solvate or solvate of a salt thereof;
with the provisio following compounds are excluded:

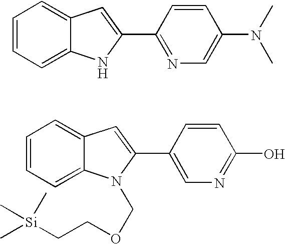

In another aspect of the invention, there is provided a compound according to formula Ib, wherein R8 is H; R10 is selected from amino, methylamino, dimethylamino and NH(CH₂)₂₋₄G7; R11 is selected from OSi(CH₃)₂C(CH₃)₃, H, fluoro, hydroxy, methoxy, Sn(C₁₋₄ alkyl)₃ and N₂⁺; R12 is selected from H, SO₂(p-methyl)phenyl, CO₂(CH₂)₂Si(CH₃)₂, CO₂t-butyl, Si(CH₃)₂C(CH₃)₃ and P(═S)phenyl₂.

In another aspect of the invention, there is provided a compound according to formula Ib, is wherein R8 is H; R9 is selected from H, fluoro and nitro; R10 is selected from amino, methylamino, dimethylamino, NH(CH₂)₂₋₄G7, N(CH₃)CHO, N(CH₃)COCH₃, N(CH₃)CO₂-t-butyl, (CO)NH₂ and O(CH₂)₂₋₄G7; R11 is selected from OSi(CH₃)₂C(CH₃)₃, H, fluoro, hydroxy, methoxy, OCH₂G4, Sn(C₁₋₄ alkyl)₃ and N₂⁺; R12 is selected from H, SO₂(p-methyl)phenyl, CO₂(CH₂)₂Si(CH₃)₂, CO₂t-butyl, Si(CH₃)₂C(CH₃)₃ and P(═S)phenyl₂.

In another aspect of the invention, there is provided a compound according to formula Ib, wherein Z is a phenyl ring, wherein X₆, X₇ and X₈ are C.

In another aspect of the invention, there is provided a compound according to formula Ib, wherein Z is a pyridine ring, wherein X₆ and X₇ are C, and wherein X₈ is N.

In another aspect of the invention, there is provided a compound according to formula Ib, wherein Z is a pyridine ring, wherein X₆ and X₈ are C, and wherein X₇ is N.

In another aspect of the invention, there is provided a compound according to formula Ib, wherein Z is a pyrimidine ring, wherein X₆ and X₈ are N, and wherein X₇ is C.

In another aspect of the invention, there is provided a compound according to formula Ib, said compound being:

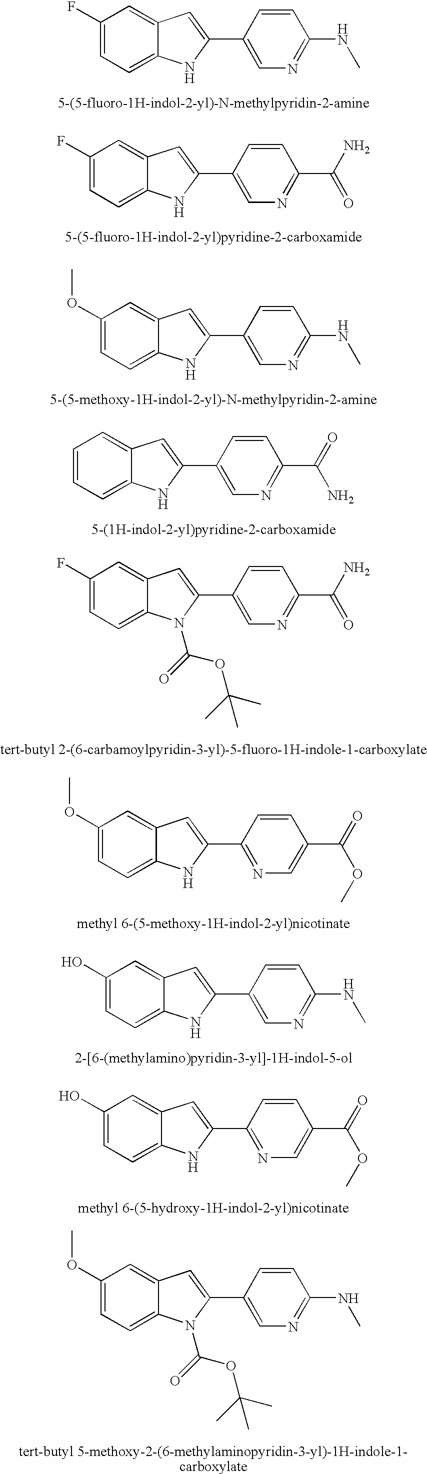

In another aspect of the invention, there is provided use of a compound according to formula Ib, as synthetic precursor in a process for preparation of a labeled compound of formula Ia, wherein said label is constituted by one [¹¹C]methyl group.

In another aspect of the invention, there is provided use of a compound according to formula Ib, as synthetic precursor in a process for preparation of a labeled compound of formula Ia, wherein said label is constituted by one ¹⁸F atom.

In another aspect of the invention, there is provided use of a compound according to formula Ib, as synthetic precursor in a process for preparation of a labeled compound of formula Ia, wherein said label is constituted by one atom selected from ¹²⁰I, ¹²³I, ¹²⁵I and ¹³¹I.

In another aspect of the invention, there is provided a pharmaceutical composition comprising a compound according to formula Ia, together with a pharmaceutically acceptable carrier.

In another aspect of the invention, there is provided a pharmaceutical composition for in vivo imaging of amyloid deposits, comprising a radio-labeled compound according to formula Ia, together with a pharmaceutically acceptable carrier.

In another aspect of the invention, there is provided an in vivo method for measuring amyloid deposits in a subject, comprising the steps of: (a) administering a detectable quantity of a pharmaceutical composition for in vivo imaging of amyloid deposits, comprising a radio-labeled compound according to formula Ia, together with a pharmaceutically acceptable carrier, and (b): detecting the binding of the compound to amyloid deposit in the subject.

In one embodiment of this aspect, said detection is carried out by the group of techniques selected from gamma imaging, magnetic resonance imaging and magnetic resonance spectroscopy.

In another embodiment of this aspect, said subject is suspected of having a disease or syndrome selected from the group consisting of Alzheimer's Disease, familial Alzheimer's Disease, Down's Syndrome and homozygotes for the apolipoprotein E4 allele.

In another aspect of the invention, there is provided compound of formula Ia, for use in therapy.

In another aspect of the invention, there is provided use of a compound of formula Ia, in the manufacture of a medicament for prevention and/or treatment of Alzheimer's Disease, familial Alzheimer's Disease, Down's Syndrome and homozygotes for the apolipoprotein E4 allele.

In another aspect of the invention, there is provided a method of prevention and/or treatment of Alzheimer's Disease, familial Alzheimer's Disease, Down's Syndrome and homozygotes for the apolipoprotein E4 allele, comprising administering to a mammal, including man in need of such prevention and/or treatment, a therapeutically effective amount of a compound of formula Ia.

### DEFINITIONS

As used herein, “alkyl”, “alkylenyl” or “alkylene” used alone or as a suffix or prefix, is intended to include both branched and straight chain saturated aliphatic hydrocarbon groups having from 1 to 12 carbon atoms or if a specified number of carbon atoms is provided then that specific number would be intended. For example “C₁₋₆ alkyl” denotes alkyl having 1, 2, 3, 4, 5 or 6 carbon atoms. When the specific number denoting the alkyl-group is the integer 0 (zero), a hydrogen-atom is intended as the substituent at the position of the alkyl-group. For example, “N(Cₒ alkyl)₂” is equivalent to “NH₂” (amino). When the specific number denoting the alkylenyl or alkylene-group is the integer 0 (zero), a bond is intended to link the groups onto which the alkylenyl or alkylene-group is substituted. For example, “NH(Cₒ alkylene)NH₂” is equivalent to “NHNH₂” (hydrazino). As used herein, the groups linked by an alkylene or alkylenyl-group are intended to be attached to the first and to the last carbon of the alkylene or alkylenyl-group. In the case of methylene, the first and the last carbon is the same. For example, “N(C₄ alkylene)”, “N(C₅ alkylene)” and “N(C₂ alkylene)₂NH” is equivalent to pyrrolidinyl, piperidinyl and piperazinyl, receptively.

Examples of alkyl include, but are not limited to, methyl, ethyl, n-propyl, i-propyl, n-butyl, i-butyl, sec-butyl, t-butyl, pentyl, and hexyl.

Examples of alkylene or alkylenyl include, but are not limited to, methylene, ethylene, propylene, and butylene.

As used herein, “alkoxy” or “alkyloxy” represents an alkyl group as defined above with the indicated number of carbon atoms attached through an oxygen bridge. Examples of alkoxy include, but are not limited to, methoxy, ethoxy, n-propoxy, isopropoxy, n-butoxy, isobutoxy, t-butoxy, n-pentoxy, isopentoxy, cyclopropylmethoxy, allyloxy and propargyloxy. Similarly, “alkylthio” or “thioalkoxy” represent an alkyl group as defined above with the indicated number of carbon atoms attached through a sulphur bridge.

As used herein, “fluoroalkyl”, “fluoroalkylene” and “fluoroalkoxy”, used alone or as a suffix or prefix, refers to groups in which one, two, or three of the hydrogen(s) attached to the carbon(s) of the corresponding alkyl, alkylene and alkoxy-groups are replaced by fluoro. Examples of fluoroalkyl include, but are not limited to, trifluoromethyl, difluoromethyl, fluoromethyl, 2,2,2-trifluoroethyl, 2-fluoroethyl and 3-fluoropropyl.

Examples of fluoroalkylene include, but are not limited to, difluoromethylene, fluoromethylene, 2,2-difluorobutylene and 2,2,3-trifluorobutylene.

Examples of fluoroalkoxy include, but are not limited to, trifluoromethoxy, 2,2,2-trifluoroethoxy, 3,3,3-trifluoropropoxy and 2,2-difluoropropoxy.

As used herein, “aromatic” refers to hydrocarbonyl groups having one or more unsaturated carbon ring(s) having aromatic characters, (e.g. 4n+2 delocalized electrons where “n” is an integer) and comprising up to about 14 carbon atoms. In addition “heteroaromatic” refers to groups having one or more unsaturated rings containing carbon and one or more heteroatoms such as nitrogen, oxygen or sulphur having aromatic character (e.g. 4n+2 delocalized electrons).

As used herein, the term “aryl” refers to an aromatic ring structure made up of from 5 to 14 carbon atoms. Ring structures containing 5, 6, 7 and 8 carbon atoms would be single-ring aromatic groups, for example, phenyl. Ring structures containing 8, 9, 10, 11, 12, 13, or 14 would be polycyclic, for example naphthyl. The aromatic ring can be substituted at one or more ring positions with such substituents as described above. The term “aryl” also includes polycyclic ring systems having two or more cyclic rings in which two or more carbons are common to two adjoining rings (the rings are “fused rings”) wherein at least one of the rings is aromatic, for example, the other cyclic rings can be cycloalkyls, cycloalkenyls, cycloalkynyls, aryls and/or heterocyclyls. The terms ortho, meta and para apply to 1,2-, 1,3- and 1,4-disubstituted benzenes, respectively. For example, the names 1,2-dimethylbenzene and ortho-dimethylbenzene are synonymous.

As used herein, the term “cycloalkyl” is intended to include saturated ring groups, having the specified number of carbon atoms. These may include fused or bridged polycyclic systems. Preferred cycloalkyls have from 3 to 10 carbon atoms in their ring structure, and more preferably have 3, 4, 5, and 6 carbons in the ring structure. For example, “C₃₋₆ cycloalkyl” denotes such groups as cyclopropyl, cyclobutyl, cyclopentyl, or cyclohexyl.

As used herein, “halo” or “halogen” refers to fluoro, chloro, bromo, and iodo. “Counterion” is used, for example, to represent a small, negatively charged species such as chloride, bromide, hydroxide, acetate, sulfate, tosylate, benezensulfonate, and the like.

As used herein, the term “heterocyclyl” or “heterocyclic” or “heterocycle” refers to a saturated, unsaturated or partially saturated, monocyclic, bicyclic or tricyclic ring (unless otherwise stated) containing 3 to 20 atoms of which 1, 2, 3, 4 or 5 ring atoms are chosen from nitrogen, sulphur or oxygen, which may, unless otherwise specified, be carbon or nitrogen linked, wherein a —CH₂— group is optionally be replaced by a —C(O)—; and where unless stated to the contrary a ring nitrogen or sulphur atom is optionally oxidised to form the N-oxide or S-oxide(s) or a ring nitrogen is optionally quarternized; wherein a ring NH is optionally substituted by acetyl, formyl, methyl or mesyl; and a ring is optionally substituted by one or more halo. It is understood that when the total number of S and O atoms in the heterocyclyl exceeds 1, then these heteroatoms are not adjacent to one another. If the said heterocyclyl group is bi- or tricyclic then at least one of the rings may optionally be a heteroaromatic or aromatic ring provided that at least one of the rings is non-heteroaromatic. If the said heterocyclyl group is monocyclic then it must not be aromatic. Examples of heterocyclyls include, but are not limited to, piperidinyl, N-acetylpiperidinyl, N-methylpiperidinyl, N-formylpiperazinyl, N-mesylpiperazinyl, homopiperazinyl, piperazinyl, azetidinyl, oxetanyl, morpholinyl, tetrahydroisoquinolinyl, tetrahydroquinolinyl, indolinyl, tetrahydropyranyl, dihydro-2H-pyranyl, tetrahydrofuranyl and 2,5-dioxoimidazolidinyl.

As used herein, “heteroaryl” refers to a heteroaromatic heterocycle having at least one heteroatom ring member such as sulfur, oxygen, or nitrogen. Heteroaryl groups include monocyclic and polycyclic (e.g., having 2, 3 or 4 fused rings) systems. Examples of heteroaryl groups include without limitation, pyridyl (i.e., pyridinyl), pyrimidinyl, pyrazinyl, pyridazinyl, triazinyl, furyl (i.e. furanyl), quinolyl, isoquinolyl, thienyl, imidazolyl, thiazolyl, indolyl, pyrryl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, benzothienyl, purinyl, carbazolyl, benzimidazolyl, indolinyl, and the like.

As used herein, the phrase “protecting group” or “protective group” means temporary substituents which protect a potentially reactive functional group from undesired chemical transformations. Examples of such protecting groups include esters of carboxylic acids, silyl ethers of alcohols, and acetals and ketals of aldehydes and ketones, respectively. A sub-group of protecting groups are those which protect a nucleophilic group (e.g. a phenolic hydroxy group or the N—H functionality of an indol-ring) against alkylation and thus permit selective N-alkylation of an amino-group present in the same molecule under basic conditions. Examples of such protecting groups include, but is not limited to, methyl, 2-(trimethylsilyl)ethoxymethyl, alkoxymethyl and t-butyldimethylsilyl.

As used herein, “pharmaceutically acceptable” is employed to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, phosphoric, and the like; and the salts prepared from organic acids such as lactic, maleic, citric, benzoic, methanesulfonic, and the like.

The pharmaceutically acceptable salts of the present invention can be synthesized from the parent compound that contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used.

As used herein, “in vivo hydrolysable precursors” means an in vivo hydrolysable (or cleavable) ester of a compound of the invention that contains a carboxy or a hydroxy group. For example amino acid esters, C₁₋₆ alkoxymethyl esters like methoxymethyl; C₁₋₆alkanoyloxymethyl esters like pivaloyloxymethyl; C₃₋₈cycloalkoxycarbonyloxy C₁₋₆alkyl esters like 1-cyclohexylcarbonyloxyethyl, acetoxymethoxy, or phosphoramidic cyclic esters.

As used herein, “tautomer” means other structural isomers that exist in equilibrium resulting from the migration of a hydrogen atom. For example, keto-enol tautomerism where the resulting compound has the properties of both a ketone and an unsaturated alcohol.

As used herein “stable compound” and “stable structure” are meant to indicate a compound that is sufficiently robust to survive isolation to a useful degree of purity from a reaction mixture, and subsequent prolonged storage in the cold or at ambient temperature, and optionally formulated into an efficacious therapeutic or diagnostic agent.

Compounds of the invention further include hydrates and solvates.

The present invention includes isotopically labeled compounds of the invention. An “isotopically-labeled”, “radio-labeled”, “labeled”, “detectable” or “detectable amyloid binding” compound, or a “radioligand” is a compound of the invention where one or more atoms are replaced or substituted by an atom having an atomic mass or mass number different from the atomic mass or mass number typically found in nature (i.e., naturally occurring). One non-limiting exception is ¹⁹F, which allows detection of a molecule which contains this element without enrichment to a higher degree than what is naturally occurring. Compounds carrying the substituent ¹⁹F may thus also be referred to as “labeled” or the like. Suitable radionuclides (i.e. “detectable isotopes”) that may be incorporated in compounds of the present invention include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. It is to be understood that an isotopically labeled compound of the invention need only to be enriched with a detectable isotope to, or above, the degree which allows detection with a technique suitable for the particular application, e.g. in a detectable compound of the invention labeled with ¹¹C, the carbon-atom of the labeled group of the labeled compound may be constituted by ¹²C or other carbon-isotopes in a fraction of the molecules. The radionuclide that is incorporated in the instant radiolabeled compounds will depend on the specific application of that radiolabeled compound. For example, for in vitro plaque or receptor labeling and competition assays, compounds that incorporate ³H, ¹⁴C, or ¹²⁵I will generally be most useful. For in vivo imaging applications ¹¹C, ¹³C, ¹⁸F, ¹⁹F, ¹²⁰I, ¹²³I, ¹³¹I, ⁷⁵Br, or ⁷⁶Br will generally be most useful.

Examples of an “effective amount” include amounts that enable imaging of amyloid deposit(s) in vivo, that yield acceptable toxicity and bioavailability levels for pharmaceutical use, and/or prevent cell degeneration and toxicity associated with fibril formation.

This invention also provides radiolabeled 2-heteroaryl substituted indole derivatives as amyloid imaging agents and synthetic precursor compounds from which such are prepared.

### Methods of Use

The compounds of the present invention may be used to determine the presence, location and/or amount of one or more amyloid deposit(s) in an organ or body area, including the brain, of an animal or human. Amyloid deposit(s) include, without limitation, deposit(s) of A13. In allowing the temporal sequence of amyloid deposition to be followed, the inventive compounds may farther be used to correlate amyloid deposition with the onset of clinical symptoms associated with a disease, disorder or condition. The inventive compounds may ultimately be used to treat, and to diagnose a disease, disorder or condition characterized by amyloid deposition, such as AD, familial AD, Down's syndrome, amyloidosis and homozygotes for the apolipoprotein E4 allele.

The method of this invention determines the presence and location of amyloid deposits in an organ or body area, preferably brain, of a patient. The present method comprises administration of a detectable quantity of a pharmaceutical composition containing an amyloid-binding compound of the present invention called a “detectable compound,” or a pharmaceutically acceptable water-soluble salt thereof, to a patient. A “detectable quantity” means that the amount of the detectable compound that is administered is sufficient to enable detection of binding of the compound to amyloid. An “imaging effective quantity” means that the amount of the detectable compound that is administered is sufficient to enable imaging of binding of the compound to amyloid.

The invention employs amyloid probes which, in conjunction with non-invasive neuroimaging techniques such as magnetic resonance spectroscopy (MRS) or imaging (MINI), or gamma imaging such as positron emission tomography (PET) or single-photon emission computed tomography (SPECT), are used to quantify amyloid deposition in vivo. The term “in vivo imaging”, or “imaging”, refers to any method which permits the detection of a labeled heteroaryl substituted indole derivative as described herein. For gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound. A “subject” is a mammal, preferably a human, and most preferably a human suspected of having dementia.

For purposes of in vivo imaging, the type of detection instrument available is a major factor in selecting a given label. For instance, radioactive isotopes and ¹⁹F are particularly suitable for in vivo imaging in the methods of the present invention. The type of instrument used will guide the selection of the radionuclide or stable isotope. For instance, the radionuclide chosen must have a type of decay detectable by a given type of instrument.

Another consideration relates to the half-life of the radionuclide. The half-life should be long enough so that it is still detectable at the time of maximum uptake by the target, but short enough so that the host does not sustain deleterious radiation. The radiolabeled compounds of the invention can be detected using gamma imaging wherein emitted gamma irradiation of the appropriate wavelength is detected. Methods of gamma imaging include, but are not limited to, SPECT and PET. Preferably, for SPECT detection, the chosen radiolabel will lack a particulate emission, but will produce a large number of photons in a 140-200 keV range.

For PET detection, the radiolabel will be a positron-emitting radionuclide, such as ¹⁸F or ¹¹C, which will annihilate to form two gamma rays which will be detected by the PET camera.

In the present invention, amyloid binding compounds/probes are made which are useful for in vivo imaging and quantification of amyloid deposition. These compounds are to be used in conjunction with non-invasive neuroimaging techniques such as magnetic resonance spectroscopy (MRS) or imaging (MRI), positron emission tomography (PET), and single-photon emission computed tomography (SPECT). In accordance with this invention, the 2-heteroaryl substituted indole derivatives may be labeled with ¹⁹F or ¹³C for MRS/MRI by general organic chemistry techniques known in the art. The compounds may also be radiolabeled with, for example, ¹⁸F, ¹¹C, ⁷⁵Br, ⁷⁶Br, or ¹²⁰1 for PET by techniques well known in the art and are described by Fowler, J. and Wolf, A. in “Positron Emisssion Tomography and Autoradiography” 391-450 (Raven Press, 1986). The compounds also may be radiolabeled with ¹²³1 and ¹³¹I for SPECT by any of several techniques known to the art. See, e.g., Kulkarni, Int. J. Rad. Appl. &Inst. (Part B) 18: 647 (1991). The compounds may also be radiolabeled with known metal radiolabels, such as Technetium-99m (^{99m}Tc). Modification of the substituents to introduce ligands that bind such metal ions can be effected without undue experimentation by one of ordinary skill in the radiolabeling art. The metal radiolabeled compound can then be used to detect amyloid deposits. Preparing radiolabeled derivatives of Tc-99m is well known in the art. See, for example, Zhuang et al. Nuclear Medicine & Biology 26(2):217-24, (1999); Oya et al. Nuclear Medicine & Biology 25(2):135-40, (1998), and Hom et al. Nuclear Medicine & Biology 24(6):485-98, (1997). In addition, the compounds may be labeled with ³H, ¹⁴C and ¹²⁵I, by methods well known to the one skilled in the art, for detection of amyloid plaque in in vitro and post mortem samples. Furthermore, fluorescent compounds of the present invention may be used for the detection of plaques present in in vitro and post mortem samples by employment of well known techniques based on the detection of fluorescence.

The methods of the present invention may use isotopes detectable by nuclear magnetic resonance spectroscopy for purposes of in vivo imaging and spectroscopy. Elements particularly useful in magnetic resonance spectroscopy include ¹⁹F and ¹³C.

Suitable radioisotopes for purposes of this invention include beta-emitters, gamma-emitters, positron-emitters, and x-ray emitters. These radioisotopes include ¹²⁰I, ¹²³I, ¹³¹I, ¹²⁵I, ¹⁸F, ¹¹C, ⁷⁵Br, and ⁷⁶Br. Suitable stable isotopes for use in Magnetic Resonance Imaging (MRI) or Spectroscopy (MRS), according to this invention, include ¹⁹F and ¹³C.

Suitable radioisotopes for in vitro quantification of amyloid in homogenates of biopsy or post-mortem tissue include ¹²⁵I, ¹⁴C, and ³H. The preferred radiolabels are ¹¹C and ¹⁸F for use in PET in vivo imaging, ¹²³I for use in SPECT imaging, ¹⁹F for MRS/MRI, and ³H and ¹⁴C for in vitro studies. However, any conventional method for visualizing diagnostic probes can be utilized in accordance with this invention.

The compounds of the present invention may be administered by any means known to one of ordinary skill in the art. For example, administration to the animal may be local or systemic and accomplished orally, parenterally, by inhalation spray, topically, rectally, nasally, buccally, vaginally, or via an implanted reservoir. The term “parenteral” as used herein includes subcutaneous, intravenous, intraarterial, intramuscular, intraperitoneal, intrathecal, intraventricular, intrasternal, intracranial, and intraosseous injection and infusion techniques.

The exact administration protocol will vary depending upon various factors including the age, body weight, general health, sex and diet of the patient; the determination of specific administration procedures would be routine to any one of ordinary skill in the art.

Dose levels on the order of about 0.001 μg/kg/day to about 10,000 mg/kg/day of an inventive compound are useful for the inventive methods. In one embodiment, the dose level is about 0.001 μg/kg/day to about 10 g/kg/day. In another embodiment, the dose level is about 0.01 μg/kg/day to about 1.0 g/kg/day. In yet another embodiment, the dose level is about 0.1 mg/kg/day to about 100 mg/kg/day.

The specific dose level for any particular patient will vary depending upon various factors, including the activity and the possible toxicity of the specific compound employed; the age, body weight, general health, sex and diet of the patient; the time of administration; the rate of excretion; the drug combination; and the form of administration. Typically, in vitro dosage-effect results provide useful guidance on the proper doses for patient administration. Studies in animal models are also helpful. The considerations for determining the proper dose levels are well known in the art and within the skills of an ordinary physician.

Any known administration regimen for regulating the timing and sequence of drug delivery may be used and repeated as necessary to effect treatment in the inventive methods.

The regimen may include pretreatment and/or co-administration with additional therapeutic agent(s).

In one embodiment, the inventive compounds are administered to an animal that is suspected of having or that is at risk of developing a disease, disorder or condition characterized by amyloid deposition. For example, the animal may be an elderly human.

In another embodiment, compounds and methods for their preparation, useful as precursors, are provided. Such precursors may be used as synthetic starting materials for the incorporation of labeled molecular fragments leading to radiolabeled 2-heteroaryl substituted indole derivatives as amyloid imaging agents.

### Method for Detecting Amyloid Deposits In Vitro

This invention further provides a method for detecting amyloid deposit(s) in vitro comprising: (i) contacting a bodily tissue with an effective amount of an inventive compound, wherein the compound would bind any amyloid deposit(s) in the tissue; and (ii) detecting binding of the compound to amyloid deposit(s) in the tissue.

The binding may be detected by any means known in the art. Examples of detection means include, without limitation, microscopic techniques, such as bright-field, fluorescence, laser-confocal and cross-polarization microscopy.

### Pharmaceutical Compositions

This invention further provides a pharmaceutical composition comprising: (i) an effective amount of at least one inventive compound; and (ii) a pharmaceutically acceptable carrier.

The composition may comprise one or more additional pharmaceutically acceptable ingredient(s), including without limitation one or more wetting agent(s), buffering agent(s), suspending agent(s), lubricating agent(s), emulsifier(s), disintegrant(s), absorbent(s), preservative(s), surfactant(s), colorant(s), flavorant(s), sweetener(s) and therapeutic agent(s).

The composition may be formulated into solid, liquid, gel or suspension form for: (1) oral administration as, for example, a drench (aqueous or non-aqueous solution or suspension), tablet (for example, targeted for buccal, sublingual or systemic absorption), bolus, powder, granule, paste for application to the tongue, hard gelatin capsule, soft gelatin capsule, mouth spray, emulsion and microemulsion; (2) parenteral administration by subcutaneous, intramuscular, intravenous or epidural injection as, for example, a sterile solution, suspension or sustained-release formulation; (3) topical application as, for example, a cream, ointment, controlled-release patch or spray applied to the skin; (4) intravaginal or intrarectal administration as, for example, a pessary, cream or foam; (5) sublingual administration; (6) ocular administration; (7) transdermal administration; or (8) nasal administration.

In one embodiment, the composition is formulated for intravenous administration and the carrier includes a fluid and/or a nutrient replenisher. In another embodiment, the composition is capable of binding specifically to amyloid in vivo, is capable of crossing the blood-brain barrier, is non-toxic at appropriate dose levels and/or has a satisfactory duration of effect. In yet another embodiment, the composition comprises about 10 mg of human serum albumin and from about 0.0005 to 500 mg of a compound of the present invention per mL of phosphate buffer containing NaCl.

The present invention further provides compositions comprising a compound of formula Ia, and at least one pharmaceutically acceptable carrier, diluent or excipient.

The present invention further provides methods of treating or preventing an Aβ-related pathology in a patient, comprising administering to the patient a therapeutically effective amount of a compound of formula Ia

The present invention further provides a compound described herein for use as a medicament.

The present invention further provides a compound described herein for the manufacture of a medicament.

Some compounds of formula Ia and Ib may have stereogenic centres and/or geometric isomeric centres (E- and Z-isomers), and it is to be understood that the invention encompasses all such optical isomers, enantiomers, diastereoisomers, atropisomers and geometric isomers.

The present invention relates to the use of compounds of formula Ia as hereinbefore defined as well as to the salts thereof. Salts for use in pharmaceutical compositions will be pharmaceutically acceptable salts, but other salts may be useful in the production of the compounds of formula Ia.

Compounds of the invention can be used as medicaments. In some embodiments, the present invention provides compounds of formula Ia, or pharmaceutically acceptable salts, tautomers or in vivo-hydrolysable precursors thereof, for use as medicaments. In some embodiments, the present invention provides compounds described here in for use as medicaments for treating or preventing an Aβ-related pathology. In some further embodiments, the Aβ-related pathology is Downs syndrome, a β-amyloid angiopathy, cerebral amyloid angiopathy, hereditary cerebral hemorrhage, a disorder associated with cognitive impairment, MCI (“mild cognitive impairment”), Alzheimer Disease, memory loss, attention deficit symptoms associated with Alzheimer disease, neurodegeneration associated with Alzheimer disease, dementia of mixed vascular origin, dementia of degenerative origin, pre-senile dementia, senile dementia, dementia associated with Parkinson's disease, progressive supranuclear palsy or cortical basal degeneration.

### Methods of Preparation

The present invention also relates to processes for preparing the compound of formula Ia and Ib as a free base, acid, or salts thereof. Throughout the following description of such processes it is to be understood that, where appropriate, suitable protecting groups will be attached to, and subsequently removed from, the various reactants and intermediates in a manner that will be readily understood by one skilled in the art of organic synthesis. Conventional procedures for using such protecting groups, as well as examples of suitable protecting groups, are described, for example, in “Protective Groups in Organic Synthesis”, 3rd ed., T. W. Green, P. G. M. Wuts, Wiley-Interscience, New York (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations”, 2nd ed., R. C. Larock, Wiley-VCH, New York (1999). References and descriptions of other suitable reactions are described in textbooks of organic chemistry, for example, “March's Advanced Organic Chemistry”, 5th ed., M. B. Smith, J. March, John Wiley & Sons (2001) or, “Organic Synthesis”, 2nd ed., M. B. Smith, McGraw-Hill, (2002). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula Ia and Ib except where defined differently. The terms “room temperature” and “ambient temperature” shall mean, unless otherwise specified, a temperature between 16 and 25° C. The term “reflux” shall mean, unless otherwise stated, in reference to an employed solvent using a temperature at or slightly above the boiling point of the named solvent. It is understood that microwaves can be used for the heating of reaction mixtures. The terms “flash chromatography” or “flash column chromatography” shall mean preparative chromatography on silica using an organic solvent, or mixtures thereof, as mobile phase.

### ABBREVIATIONS

Ac acetate;
atm atmosphere;
aq. aqueous;
Boc t-butoxycarbonyl;
DBU 1,8-diazobicyclo[5.4.0]undec-7-ene
DCM dichloromethane;

### DMA N,N-dimethylacetamide;

DME 1,2-dimethoxyethane;

### DMF N,N-dimethylformamide;

DMSO dimethyl sulfoxide;
dppf 1,1′-bis(diphenylphosphino)ferrocene;
EtOAc ethyl acetate;
EtOH ethanol;
Et₂O diethylether;
h hour(s);
hep heptane;
hex hexane(s);
MeCN acetonitrile;
MeOH methanol;
o.n. over night;
Pd(dba)₂ bis(dibenzylideneacetone)palladium(0);
Pd(dppf)Cl₂ 1,1′-bis(diphenylphosphino)ferrocene)dichloropalladium(II);
Pd(PPh₃)₂Cl₂ dichlorobis(triphenylphosphine)palladium;
prep. HPLC preparative HPLC;
PTSA p-toluenesulfonic acid;
r.t. room temperature;
r.m. reaction mixture;
sat. saturated;
TBAF tetrabutylammonium fluoride;
TFA trifluoroacetic acid;
THF tetrahydrofurane;
Tos tosylate
OTf trifluoromethanesulfonate

### Preparation of Intermediates

Compounds of formula II and III are useful intermediates in the preparation of compound of formula Ia and Ib. The synthesis of indoles are well known and suitable examples are described in textbooks of organic chemistry, for example, “March's Advanced Organic Chemistry”, 5th ed., M. B. Smith, J. March, John Wiley & Sons (2001) or, Organic Chemistry, Clayden, Greeves, Warren, Wothers (2001). Compounds of formula II and III are either commercially available, or can be prepared from either commercially available, or in the literature described compounds. For example, compounds in which one or more of Y₁, Y₂, R1, R2, R3, R4, R5 does not correspond to the definitions of formula II or III, can be used for the preparation of compounds of formula II or III by transformations or introduction of substituents or groups. Such examples are given below:

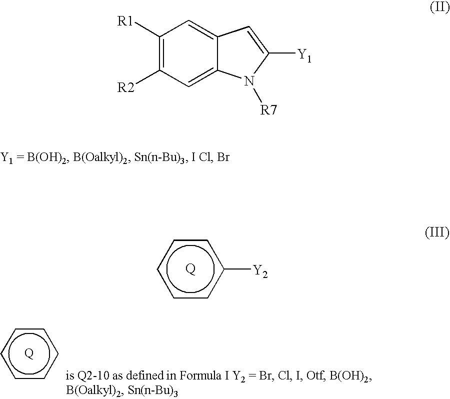

1) Preparation of Compounds of Formula II in which Y₁ is B(Oalkyl)₂ or B(OH)₂:

From the corresponding chlorides, bromides, iodides or triflates through palladium catalysed borylation employing for example bis(pinacolato)diboran or dialkoxyboranes as reagents under palladium catalysis, using for example PdCl₂(dppf), or Pd(dba)₂ with added tricyclohexylphosphine, as catalysts, together with stoichiometric amounts of a base such as KOAc and NEt₃ in solvents such as DMSO, DMF, DMA, THF or dioxan at a temperature from r.t. to 80° C., alternatively subsequently followed by acidic hydrolysis (Ishiyama et al. Tetrahedron 2001, 57, 9813; Murata et al. J. Org. Chem. 2000, 65, 164).

2) Preparation of Compounds of Formula II in which Y₁ is Halogen:

a) Halogenation at the 2-position of indoles can be achieved by N-protection of the indole, with for example Boc₂O, followed by treatment with BuLi and I₂ to afford the 2-iodo-indole (Roy et al. Tetrahedron Lett. 2005, 46, 1325-1328).

b) By Katritzkys method of using BuLi and then CO₂ as an activating and N-protecting group (Katritzky et al. Tetrahedron Lett. 1985, 26, 5935-5938) followed by the introduction of an electrophile such as hexachloroethane, 1,2-dibromo-tetrachloroethane or 1,2-diiodoethane (Bergman et al. J. Org. Chem., 1992, 57, 2495-2497).

### Methods of Preparation of Non-Labeled Compounds of Formula Ia and Ib

Non-limiting examples of methods for the preparation of compounds of formula Ia and Ib are given below:

### 1) Preparation by Palladium-Catalyzed Cross-Coupling of Intermediates II and III:

a) Palladium-catalyzed Suzuki- or Stille coupling of aryl halides, or pseudo-halides, of intermediates of formula III (e.g. Y₂=chloride, bromide, iodide or triflate) with boronic acids or esters of formula II (e.g. Y₁=B(OH)₂ or B(Oalkyl)₂), or stannanes of formula II (e.g. Y₁═Sn(n-Bu)₃). A palladium catalyst such as Pd(dppf)Cl₂ may be used in a solvent such as DMF or THF at a temperature of e.g. 80° C. (Kotha et al. Tetrahedron 2002, 58, 9633-9695; Suzuki J. Organomet. Chem. 1999, 576, 147-168; Fugami et al. Top. Curr. Chem. 2002, 219, 87-130.)

b) The palladium-catalysed Suzuki- or Stille coupling reaction could also be carried out where Y₁ is swapped with Y₂ i.e. where the indole moiety of formula II carry the halogen and the boronic acid or ester is attached to the intermediate of formula III.

### Methods of Preparation of Labeled Compounds of Formula Ia

In general, the same synthetic reactions used for the assembly of non-labeled compounds of formula Ia from non-labeled reagents or intermediates, can be employed for the analogous incorporation of a detectable isotope by use of the corresponding labeled reagents or intermediates.

It is preferred to introduce the label at a late stage of the synthesis toward compounds of formula Ia, especially if the label is an isotope with relatively short half-life, such as ¹¹C. Most preferred is to do this introduction as the last synthetic step. Several useful reagents, synthons or intermediates labeled with long-lived or non-radioactive isotopes, including for example [^{2/3}H]H₂, [^{2/3}H]CH₃I, [^{13/14}C]CH₃I, [^{13/14}C]CN⁻, [^{13/14}C]CO₂ are commercially available and can, if needed, be further synthetically transformed by conventional synthetic methods. Reagents labeled with relatively more short-lived isotopes, such as ¹¹C and ¹⁸F, are generated by a cyclotron, followed by suitable trapping and optionally further synthetic manipulations to provide the desired reagent. The generation and the synthetic manipulations of labeled reagents and intermediates, and the use and chemistries of these precursors for the synthesis of more complex labeled molecules, is well known to the one skilled in the art of radio-synthesis and labeling and is reviewed in the literature (Ungstrom et al. Acta Chem. Scand. 1999, 53, 651). For additional references see for example: Ali et al. Synthesis 1996, 423 for labeling with halogens; Antoni G., Kihlberg T., and Långström B. (2003) Handbook of nuclear chemistry, edited by Vertes A., Nagy S., and Klenscar Z., Vol. 4, 119-165 for labeling for PET-applications; Saljoughian et al. Synthesis 2002, 1781 for labeling with ³H; McCarthy et al. Curr. Pharm. Des. 2000, 6, 1057 for labeling with ¹⁴C.

Detectable isotopes, useful for the labeling of compounds of formula Ia as defined herein include, for use in PET: ¹¹C, ¹⁸F, ⁷⁵Br, ⁷⁶Br and ¹²⁰I, for use in SPECT: ¹²³I and ¹³¹I, for MRI-applications: ¹⁹F and ¹³C, for detection in in-vitro and post-mortem samples: ³H, ¹⁴C and ¹²⁵I. The most useful isotopes for labeling are ¹¹C, ¹⁸F, ¹²³I, ¹⁹F, ³H and ¹⁴C.

Below follow non-limiting descriptions on processes for the preparation of labeled compounds of formula Ia:

Compounds of formula Ia and Ib, which carry a hydroxy-, amino- or aminoalkyl group are useful precursors for O- and N-alkylation, respectively, with a labeled alkylating agent, such as [¹¹C]methyl iodide or triflate, as described in for example Solbach et al. Applied Radiation and Isotopes 2005, 62, 591 and Mathis et al. J. Med. Chem. 2003, 46, 2740, or [³H]-methyl iodide, or [¹⁴C]-methyl iodide.

For example, the compounds of formula Ia, in which one of R1 and R2 is hydroxy (the other is hydrogen), or compounds of formula Ib, in which one of R8 and R11 is hydroxy (the other is hydrogen), or constitute precursors for labeling. When such a precursor is treated with [¹¹C]methyl iodide under basic condition, such as in the presence of potassium carbonate, in a solvent such as DMSO, selective O-alkylation occurs in the presence of N-nucleophiles, such as amino, aminomethyl or the indole N—H functionality, because of relatively higher reactivity of the oxygen-atom after deprotonation, and thus in the formation of compounds of formula Ia and Ib in which the OH-group has been transformed into the O[¹¹C]CH₃-group. Compounds of formula Ib in which R8 or R11 is a protected (e.g. with TBDMS) hydroxy group, X₈ is N, and R10 is hydroxy, are useful precursors for labeling through O-alkylation by use of ¹¹C-methyl iodide in the presence of Ag₂CO₃ as a base (Shinzo K. Synth Comm 2006, 36, 1235).

The most preferred precursors for labeling by selective introduction of a ¹¹C-methyl group by N-alkylation, are compounds in which the reactivity to alkylation, of a present competing nucleophilic functional group, such as hydroxy or the indole N—H functionality, is lowered or blocked by a suitable protective group. The function of the protective group is, in this context, to protect the nucleophilic functional group from alkylation and should preferrably be stable under non-aqueous basic conditions, under which the desired N-alkylation is facilitated, but readily removed by other means after fulfillment of its duty. Such protective groups, and methods for their introduction and removal, are well known to the one skilled in the art. Examples of protective groups useful for protection of aromatic hydroxy-groups against competing alkylation include, but is not limited to, methyl, 2-(trimethylsilyl)ethoxymethyl, alkoxymethyl and t-butyldimethylsilyl. Removal of such a protective group after the alkylation is well known to the one skilled in the art and include, in the case of silyl-based protective groups such as t-butyldimethylsilyl, for example treatment with a fluoride ion source, such as TBAF, or treatment with water under basic conditions in a suitable solvent, such as DMSO in the presence of KOH at rt. Examples of protective groups useful for protection of the indole N—H functionality against competing alkylation include, but is not limited to, SO₂N(CH₃)₂, SO₂(p-methyl)phenyl, CO₂CH₂CCl₃, CO₂(CH₂)₂Si(CH₃)₂, t-butyldimethylsilyl and P(═S)phenyl₂. In the case where an aromatic hydroxy-functionality, and the indole N—H functionality, are simultaneously protected against alkylation, it is preferred to use one protective group, such as t-butyldimethylsilyl, or two different protective groups, which allow simultaneous de-protection of both functionalities in one laboratory step by employment of one de-protection reagent.

Compounds of formula Ia or Ib, carrying an aromatic amino-group, are useful precursor for labeling by initial diazotation (i.e. transformation of the amino-group into the N₂⁺ moiety), when appropriate, followed by conversion into the corresponding triazine derivative before subsequent treatment with labeled nucleophilic reagents according to standard reactions. Detectable isotopes that may be introduced this way include, but is not limited to ¹⁸F, ⁷⁵Br, ¹²³I, ¹²⁵I and ¹³¹I as described in for example Zhu et al. J. Org. Chem. 2002, 67, 943; Maeda et al. J. Label Compd Radiopharm 1985, 22, 487; Berridge et al. J. Label Compd Radiopharm 1985, 22, 687; Suchiro et al. J. Label Compd Radiopharm 1987, 24, 1143; Strouphauer et al. Int. J. Appl. Radiat. Isot. 1984, 35, 787; Kortylevicz et al. J. Label Compd Radiopharm 1994, 34, 1129; Khalaj et al. J. Label Compd Radiopharm 2001, 44, 235 and Rzeczotarski et al. J. Med. Chem. 1984, 27, 156.

In compounds of formula Ib, carrying an aromatic trialkyltin-group, halogenation with labeled reagents results in displacement of the trialkyltin-group as described in for example Staelens et al. J. Label Compd Radiopharm 2005, 48, 101; Hocke et al. Bioorg. Med. Chem. Lett. 2004, 14, 3963; Zhuang et al. J. Med. Chem. 2003, 46, 237; Füchtner et al. Appl. Rad. Isot. 2003, 58, 575 and Kao et al. J. Label Compd Radiopharm 2001, 44, 889. The same precursors are also useful for palladium-catalyzed conversion into the corresponding ¹¹C-labeled ketones and methyl-derivatives as described in for example Lidström et al. J. Chem. Soc. Perkin Trans. 1 1997, 2701 and Tarkiainen et al. J. Label Compd Radiopharm 2001, 44, 1013. The trialkyltin substituted compounds, in turn, are preferably prepared from the corresponding halides or pseudo-halides, such as the triflates, by well known methods employing palladium as catalyst in reaction with the corresponding distannane. When this methodology is used, the trialkyltin-group is preferably trimethyltin or tributyltin.

Compounds of formula Ib, which are carrying an aromatic trialkyltin group, preferably nBu₃Sn, X6 is carbon, X7 or X8 is nitrogen (the other is carbon), and R10 is methylamino, dimethylamino or methoxy, are suitable precursors for labeling with ¹²³I or ¹²⁵I by iododestannylation under oxidative conditions in the presence of labelled iodide according to the method described in, for example, in Zhuang et al. Nucl. Med. Biol. 2001, 28, 887.

When any one of the heterocyclic substituents in a precursor, is a leaving group suitable for nucleophilic aromatic substitution, a labeled nucleophile, such as a halogenide or cyanide, can be introduced by such a displacement resulting in a labeled compound of formula Ia, as described in for example Zhang et al. Appl. Rad. Isot. 2002, 57, 145. The aromatic ring on which the displacement takes place is preferably relatively electron-poor for a facile reaction, and might therefore need to be substituted with an electron-withdrawing activating group such as cyano, carbaldehyde or nitro. Useful reactions, closely related to nucleophilic aromatic substitutions and well known to the one skilled in the art, include the employment of stoichiometric amounts of copper-salts for the introduction of a labeled iodo-atom, and the use of palladium-catalysis for the introduction of a ¹¹C-labelled cyano-group, as described in for example Musacio et al. J. Label Compd Radiopharm 1997, 34, 39 and Andersson et al. J. Label Compd Radiopharm 1998, 41, 567 respectively. Also, an ¹⁸F-atom may be introduced, for example by use of K[¹⁸F]-K₂₂₂ in DMSO under microwave irradiation as described in Karramkam, M. et al. J. Labelled Compd. Rad. 2003, 46, 979. If the aromatic ring onto which the leaving group is positioned is more electron-deficient as compared to benzene, such as in 2-halo pyridines and pyrimidines, it is generally not needed to employ activating groups for electrophilic aromatic substitution to take place.

Compounds of formula Ia, where Q is Q2, and Ib, where R3 and R10, respectively, are either of the leaving-groups fluoro, chloro, bromo, iodo, or a sulphonate ester, and either or both of X2 and X4, and X6 and X8 is nitrogen, are suitable precursors for labeling via nucleophilic aromatic substitution. It is furthermore preferable to use a leaving group that is chemically diverse from the group introduced by the reaction with the labeled nucleophile in order to facilitate chromatographic separation of the labeled reaction product from the unconsumed precursor.

Compounds of formula Ib, in which R8 or R11 is a protected (e.g. TBDMS) hydroxy group (the other is hydrogen), R12 is not H, and R10 is O(CH₂)₂OTos or NH(CH₂)₂OTos are useful precursors for labeling with fluorine by use of either kryptofix 2.2.2-[¹⁸F]fluoride complex (Schirrmacher et al. J. Labelled Compd. Rad. 2001, 44, 627), or tetrabutylammonium [¹⁸F]fluoride in CH₃CN under heating (Hamacher et al. Appl. Radiat. Isotopes 2002, 57, 853), as sources of nucleophilic ¹⁸F for nucleophilic replacement of the formal leaving group OTos⁻. Other suitable leaving groups that may be employed are well known to the one skilled in the art and include, but is not limited to bromo, iodo, OSO₂CF₃, OSO₂CH₃ and OSO₂phenyl.

Compounds of formula Ib, in which R8 is H, R11 is OSi(G3)₃ or OCH₂G4, R10 is N(CH₃)CHO, N(CH₃)COCH₃, N(CH₃)CO₂-t-butyl or CONH₂ and R9 is nitro, N(CH₃)₃⁺, bromo, iodo, chloro are useful precursors for labeling with fluorine by use of kryptofix 2.2.2-[¹⁸F]fluoride complex as source of nucleophilic ¹⁸F for nucleophilic replacement of the formal leaving groups R9 (F. Dolle, Curr. Pharm. Design 2005, 11, 3221-3235).

Additional useful methods, well known to the one skilled in the art, for preparation of labeled compounds of formula Ia by functional group transformations of suitable precursors include N-acylation of amines with [¹¹C], [¹⁴C], or [³H]acyl chlorides, palladium-catalyzed [¹¹C] or [¹⁴C] cyanation of aromatic chlorides, bromides or iodides, transition-metal catalyzed substitution of suitable halides for ³H in the presence of [³H]H₂, and palladium-catalyzed carbonylations with [^{11/14}C]CO (Perry et al. Organometallics 1994, 13, 3346).

### Compound Examples

Below follows a number of non-limiting examples of compounds of the invention. All of the below exemplified compounds, or their corresponding non-labeled analogs, which are not solely precursors and thus indicated to be such, display an ICₛₒ of less than 20 μM in the competition binding assay described herein.

### General Methods

All solvents used were analytical grade and commercially available anhydrous solvents were routinely used for reactions. Reactions were typically run under an inert atmosphere of nitrogen or argon.

¹H spectra were recorded on a Bruker av400 NMR spectrometer, operating at 400 MHz for proton, equipped with a 3 mm flow injection SEI ¹H/D-¹³C probehead with Z-gradients, using a BEST 215 liquid handler for sample injection, or on a Bruker DPX400 NMR spectrometer, operating at 400 MHz for proton, equipped with a 5 mm 4-nucleus probehead equipped with Z-gradients.

Unless specifically noted in the examples, ¹H spectra were recorded at 400 MHz in DMSO-d₆ as solvent. The residual solvent signal was used as reference. The following reference signals were used: the middle line of DMSO-d₆ δ 2.50; the middle line of CD₃OD δ 3.31; CDCl₃ δ 7.26. In those instances where spectra were run in a mixture of CDCl₃ and CD₃OD, the reference was set to 3.31 ppm. All chemical shifts are in ppm on the delta-scale (δ) and the fine splitting of the signals as appearing in the recordings (s: singlet, d: doublet, t: triplet, q: quartet, m: multiplet, br: broad signal).

³H spectra were recorded on a Bruker DRX600 NMR Spectrometer, operating at 640 MHz for tritium and at 600 MHz for proton, equipped with a 5 mm ³H/¹H SEX probehead with Z-gradients. ¹H decoupled ³H spectra were recorded on samples dissolved in CD₃OD. For ³H NMR spectra referencing, a ghost reference frequency was used, as calculated by multiplying the frequency of internal TMS in a ¹H spectrum with the Larmor frequency ratio between ³H and ¹H (1.06663975), according to the description in Al-Rawi et al. J. Chem. Soc. Perkin Trans. II 1974, 1635.

Mass spectra were recorded on a Waters LCMS consisting of an Alliance 2795 or Acquity system (LC), Waters PDA 2996, and ELS detector (Sedex 75) and a ZMD single quadrupole or ZQ mass spectrometer. The mass spectrometer was equipped with an electrospray ion source (ES) operated in a positive or negative ion mode. The capillary voltage was 3 kV and cone voltage was 30 V. The mass spectrometer was scanned between m/z 100-600 with a scan time of 0.7 s. The column temperature was set to 40° C. (Alliance) or 65° C. (Acquity). A linear gradient was applied starting at 100% A (A: 10 mM NH₄OAc in 5% MeCN) and ending at 100% B (B: MeCN). The column used was a X-Terra MS C8, 3.0×50; 3.5 μm (Waters) run at 1.0 mL/min (Alliance), or an Acquity UPLC™ BEH C₈ 1.7 μm 2.1×50 mm run at 1.2 mL/min.

Preparative chromatography (prep. HPLC) was run on either of two Waters autopurification HPLCs: (1) equipped with a diode array detector and an XTerra MS C8 column, 19×300 mm, 10 μm. (2) consisting of a ZQ mass spectrometer detector run with ESI in positive mode at a capillary voltage of 3 kV and a cone voltage of 30 V, using mixed triggering, UV and MS signal, to determine the fraction collection. Column: XBridge™ Prep C8 5 μm OBD™ 19×100 mm. Gradients with MeCN/(95:5 0.1M NH₄OAc:MeCN) were used at a flow rate of 20 or 25 mL/min.

Microwave heating was performed in a Creator, Initiator or Smith Synthesizer Single-mode microwave cavity producing continuous irradiation at 2450 MHz.

### Compounds and Precursors

Below follows a number of non-limiting examples of compounds of the invention. All of the below exemplified compounds, or their corresponding non-labeled analogs, which are not solely precursors and thus indicated to be intermediates in the examples below, display an IC₅₀ of less than 20 μM in the competition binding assay described herein.

### EXAMPLE 1

### 5-(5-Fluoro-1H-indol-2-yl)—N-methylpyridin-2-amine

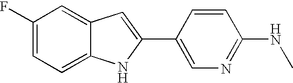

### a) tert-butyl 5-fluoro-2-[6-(methylamino)pyridin-3-yl]-1H-indole-1-carboxylate

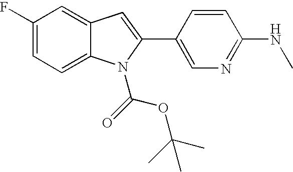

1-(tert-butoxycarbonyl)-5-fluoroindole-2-boronic acid (1 mmol), 5-bromopyridine-2-methylamine (1 mmol), Pd(dppf)Cl₂ (0.05 mmol) and 2M Na₂CO₃ (aq.) (1.5 mL) were mixed in THF/water 5:1 (10 mL) in a 20 mL microwave vial. The reaction mixture was stirred at 120° C. in the microwave reactor for 15 min. Water was added and the solution was extracted with EtOAc. The organic extracts were dried over Na₂SO₄, filtered and concentrated. The crude mixture was purified by flash chromatography (Heptane/EtOAc gradient) to give the title intermediate (114 mg); MS m/z (M+H) 342.

### b.) 5-(5-Fluoro-1H-indol-2-yl)-N-methylpyridin-2-amine (title compound)

tert-butyl 5-fluoro-2-[6-(methylamino)pyridin-3-yl]-1H-indole-1-carboxylate (114 mg, 0.33 mmol) was diluted in DCM (10.0 mL) and TFA (1.0 mL) was slowly added. The mixture was stirred at 20° C. for 15 h. NaHCO₃ (sat. aq.) (50 mL) was added followed by EtOAc. After separation, the organic layer was dried over Na₂SO₄, filtered and evaporated under vacuum. The crude product was purified by preparative HPLC to afford the title compound (40 mg). ¹H NMR δ ppm 11.39 (s, 1H) 8.50 (d, 1H) 7.82 (dd, 1H) 7.31 (dd, 1

H) 7.20 (dd, 1H) 6.78-6.93 (m, 1H) 6.71 (d, 1H) 6.65 (d, 1H) 6.53 (d, 1H) 2.82 (d, 4H); MS m/z (M+H) 242.

### EXAMPLE 2

### tert-Butyl 2-(6-carbamoylpyridin-3-yl)-5-fluoro-1H-indole-1-carboxylate

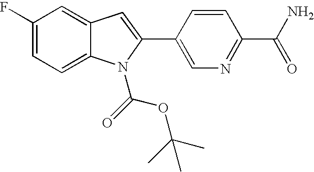

1-(tert-Butoxycarbonyl)-5-fluoroindole-2-boronic acid (1 mmol), 5-bromopyridine-2-carboxamide (1 mmol), Pd(dppf)Cl₂ (0.05 mmol) and 2M Na₂CO₃ (aq.) (1.5 mL) solution were mixed in THF/water 5:1 (10 mL) in a 20 mL microwave vial. The reaction mixture was stirred at 120° C. in the microwave reactor for 15 min. Water was added and the solution was extracted with EtOAc. The organic extracts were dried over Na₂SO₄, filtered and concentrated to afford a crude mixture which was purified by flash chromatography (Heptane/EtOAc gradient) to give the title compound (200 mg). ¹H NMR δ ppm 8.75 (s, 1H) 8.12-8.23 (m, 2H) 8.07-8.13 (m, 2H) 7.68 (br. s., 1H) 7.48 (dd, 1H) 7.15-7.32 (m, 1H) 6.92 (s, 1H) 1.30 (s, 9H); MS m/z (M+H) 356.

### EXAMPLE 3

### 5-(5-fluoro-1H-indol-2yl)pyridine-2-carboxamide

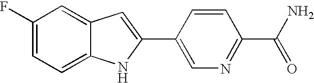

tert-Butyl 2-(6-carbamoylpyridin-3-yl)-5-fluoro-1H-indole-1-carboxylate (0.48 mmol) was diluted in DCM (10.0 mL) and TFA (1.0 mL) was slowly added. The mixture was stirred at 20° C. for 15 h. NaHCO₃ (sat. aq.) (50 mL) was added followed by EtOAc. After separation, the organic layer was dried over Na₂SO₄, filtered and evaporated under vacuum. The crude product was purified by preparative HPLC to give the title compound (64 mg). ¹H NMR δ ppm 11.70 (br. s., 1H) 9.09 (d, 1H) 8.30 (dd, 1H) 8.07 (d, 1H) 7.37 (d, 1H) 7.22 (s, 1H) 7.08 (d, 1H) 6.83 (dd, 1H) 3.90 (s, 3H) 3.77 (s, 3H); MS m/z (M−H) 254.

### EXAMPLE 4

### 5-(5-Methoxy-1H-indol-2-yl)—N-methylpyridin-2-amine

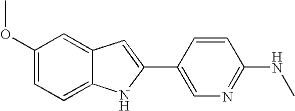

### a) tert-butyl 5-methoxy-2-[6-(methylamino)pyridin-3-yl]1H-indole-1-carboxylate

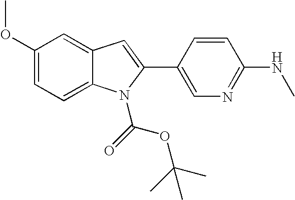

1-(tert-Butoxycarbonyl)-5-methoxyindole-2-boronic acid (2 mmol), 5-bromopyridine-2-methylamine (2 mmol), Pd(dppf)Cl₂ (0.10 mmol) and 2M Na₂CO₃ (aq.) (3 mL) were mixed in THF/water 5:1 (10 mL) in a 20 mL microwave vial. The reaction mixture was stirred at 120° C. in the microwave reactor for 15 min. Water was added and the solution was extracted with EtOAc. The organic extracts were dried over Na₂SO₄, filtered and concentrated. The crude mixture was purified by flash chromatography (Heptane/EtOAc gradient) to afford the title intermediate (256 mg). ¹H NMR δ ppm 8.04 (d, 1H) 7.94 (d, 1H) 7.43 (dd, 1H) 7.08 (d, 1H) 6.89 (dd, 1H) 6.64 (d, 1H) 6.53 (s, 1H) 6.50 (d, 1H) 3.78 (s, 3H) 2.80 (d, 3H) 1.36 (s, 9H); MS m/z (M+H) 354.

### b) 5-(5-Methoxy-1H-indol-2-yl)-N-methylpyridin-2-amine (Title Compound)

tert-butyl 5-methoxy-2-[6-(methylamino)pyridin-3-yl]-1H-indole-1-carboxylate (0.64 mmol) was diluted in DCM (10.0 mL) and TFA (1.0 mL) was slowly added. The mixture was stirred at 20° C. for 15 h. NaHCO₃ (sat. aq.) (50 mL) was added followed by EtOAc. After separation, the organic layer was dried over Na₂SO₄, filtered and evaporated under vacuum. The crude product was purified by preparative HPLC to give the product (101 mg). ¹H NMR δ ppm 11.12 (s, 1H) 8.47 (d, 1H) 7.79 (dd, 1H) 7.22 (d, 1H) 6.96 (d, 1H) 6.60-6.75 (m, 2H) 6.57 (d, 1H) 6.52 (d, 1H) 3.74 (s, 3H) 2.81 (d, 3H); MS m/z (M+H) 254.

### EXAMPLE 5

### 5-(1H-Indol-2-yl)pyridine-2-carboxamide

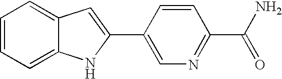

### a) tert-Butyl 2-(6-carbamoylpyridin-3-yl)-1H-indole-1-carboxylate

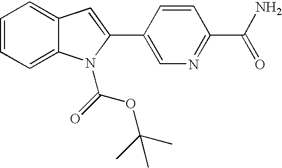

1-(tert-butoxycarbonyl)indole-2-boronic acid (1 mmol), 5-bromopyridine-2-carboxamide (1 mmol), Pd(dppf)Cl₂ (0.05 mmol) and aq 2M Na₂CO₃ (1.5 mL) solution were mixed in THF/water 5:1 (10 mL) in a 20 mL microwave vial. The reaction mixture was stirred at 120° C. in the microwave reactor for 15 min. Water was added and the solution was extracted with EtOAc. The organic extracts were dried over Na₂SO₄, filtered and concentrated. The crude mixture was purified by flash chromatography (Heptane/EtOAc gradient) to afford the title intermediate (172 mg). MS m/z (M+H) 338.

### b) 5-(1H-Indol-2-yl)pyridine-2-carboxamide (Title Compound)

tert-Butyl 2-(6-carbamoylpyridin-3-yl)-1H-indole-1-carboxylate (0.51 mmol) was diluted in DCM (10.0 mL) and TFA (1.0 mL) was slowly added. The mixture was stirred at 20° C. for 15 h. NaHCO₃ (sat. aq.) (50 mL) was added followed by EtOAc. After separation, the organic layer was dried over Na₂SO₄, filtered and evaporated under vacuum. The crude product was purified by preparative HPLC to afford the title compound (56 mg). ¹H NMR δ ppm 11.80 (s, 1H) 9.13 (d, 1H) 8.39 (dd, 1H) 7.95-8.19 (m, 2H) 7.53-7.72 (m, 2H) 7.45 (d, 1H) 7.11-7.26 (m, 2H) 6.90-7.09 (m, 1H); MS m/z (M+H) 238.

### EXAMPLE 6

### Methyl 6-(5-methoxy-1H-indol-2-yl)-nicotinate

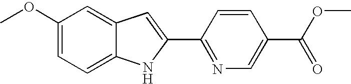

### a) tert-butyl 5-methoxy-2-[5-(methoxycarbonyl)pyridin-2-yl]-1H-indole-1-carboxylate

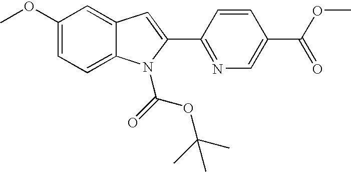

1-(tert-butoxycarbonyl)-5-methoxyindole-2-boronic acid (2 mmol), 6-bromo-nicotinic acid methyl ester (2 mmol), Pd(dppf)Cl₂ (0.10 mmol) and 2M Na₂CO₃ (aq.) (3 mL) were mixed in THF/water 5:1 (10 mL) in a 20 mL microwave vial. The reaction mixture was stirred at 120° C. in the microwave reactor for 15 min. Water was added and the solution was extracted with EtOAc. The organic extracts were dried over Na₂SO₄, filtered and concentrated to afford a crude mixture which was purified by flash chromatography (Heptane/EtOAc gradient) to give the title intermediate (397 mg). ¹H NMR (400 MHz, DMSO-d₆) δ ppm 8.99-9.21 (m, 1H) 8.38 (dd, 2H) 7.93 (d, 2H) 7.80-7.89 (m, 2H) 7.20 (d, 1H) 6.97-7.11 (m, 2H) 3.92 (s, 3H) 3.81 (s, 3H) 1.27 (s, 9H); MS m/z (M+H) 383.

### b) Methyl 6-(5-methoxy-1H-indol-2-yl)-nicotinate (Title Compound)

tert-butyl 5-methoxy-2-[5-(methoxycarbonyl)pyridin-2-yl]-1H-indole-1-carboxylate (1.04 mmol) was diluted in DCM (15.0 mL) and TFA (1.5 mL) was slowly added. The mixture was stirred at 20° C. for 15 h. NaHCO₃ (sat. aq.) (50 mL) was added followed by EtOAc. After separation, the organic layer was dried over Na₂SO₄, filtered and evaporated under vacuum. The crude product was purified by preparative HPLC to afford the title compound (153 mg). ¹H NMR δ ppm 11.70 (br. s., 1H) 9.09 (d, 1H) 8.30 (dd, 1H) 8.07 (d, 1H) 7.37 (d, 1H) 7.22 (s, 1H) 7.08 (d, 1H) 6.83 (dd, 1H) 3.90 (s, 3H) 3.77 (s, 3H); MS m/z (M+H) 283.

### EXAMPLE 7

### 2-[6-(Methylamino)pyridin-3-yl]-1H-indol-5-ol

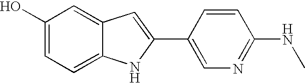

5-(5-Methoxy-1H-indol-2-yl)—N-methylpyridin-2-amine (0.34 mmol) was dispersed in DCM (3 mL) at 0° C. under argon atmosphere. 1M BBr₃ in DCM (1.3 mL) was slowly added and the mixture was stirred at 0° C. for 20 min. The ice bath was removed and the mixture was stirred for 2 h. Water was added dropwise to the solution, followed by NaHCO₃ (sat. aq.). The aqueous layer was extracted with EtOAc. The organic layer was dried over Na₂SO₄, filtered and evaporated under vacuum. The crude product was purified by preparative HPLC to give the title compound (15 mg). ¹H NMR δ ppm 10.96 (s, 1H) 8.56 (s, 1H) 8.45 (d, 1H) 7.77 (dd, 1H) 7.12 (d, 1H) 6.77 (d, 1H) 6.62 (d, 1H) 6.40-6.58 (m, 3H) 2.81 (d, 3H); MS m/z (M+H) 240.

### EXAMPLE 8

### 6-(5-Hydroxy-1H-indol-2-yl)-Nicotinic Acid Methyl Ester

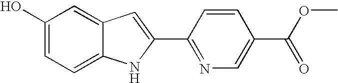

Methyl 6-(5-methoxy-1H-indol-2-yl)-nicotinate (200 mg, 0.71 mmol) was dispersed in DCM (5 mL) at 0° C. under argon atmosphere. BBr₃ 1M in DCM (3.0 mL) was slowly added and the mixture was stirred at 0° C. for 20 min. The ice bath was removed and the mixture was stirred for 2 h. Water was added dropwise to the solution, followed by sat. aq. solution of NaHCO₃. The aqueous layer was extracted with EtOAc. The organic layer was dried over Na₂SO₄, filtered and evaporated under vacuum. The crude product was purified by preparative HPLC to give the title compound (9 mg). ¹H NMR δ ppm 11.55 (s, 1H) 8.93-9.22 (m, 1H) 8.79 (s, 1H) 8.28 (dd, 1H) 8.04 (d, 1H) 7.27 (d, 1H) 7.12 (d, 1H) 6.88 (d, 1H) 6.71 (dd, 1H) 3.90 (s, 3H); MS m/z (M+H) 269.

### BIOLOGICAL EXAMPLES

The following compounds were used as comparative compounds and are referred to in the text below by their indicated corresponding names.

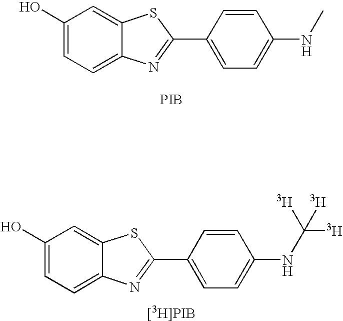

Compounds of the present invention were tested in one or several of the following assays/experiments/studies:

### Competition Binding Assay

Competition binding was performed in 384-well FB filter plates using synthetic Aβ 1-40 in 2.7 nM of [³H]PIB (or another ³H-labeled radioligand when so mentioned) in phosphate buffer at pH 7.5, by adding various concentrations of non-radioactive compounds originally dissolved in DMSO. The binding mixture was incubated for 30 min at room temperature, followed by vacuum filtration, and subsequently by washing twice with 1% Triton-X100. Scintillation fluid was thereafter added to the collected Aβ 1-40 on the filter plate, and the activity of the bound remaining radioligand ([³H]PIB or another ³H-labeled radioligand) was measured using 1450 Microbeta from PerkinElmer.

### Dissociation Experiments

Dissociation experiments were performed in 96-well polypropylene deep well plates. 2 μM human synthetic Aβ 1-40 fibrils in phosphate buffer pH 7.5, or buffer alone as control, was incubated with 9 nM of a ³H-labeled radioligand of the present invention for 4 h at room temperature. Dissociation was started at different time points, by the addition of an equal volume of a non-labeled compound of the present invention, or a reference compound (10 μM), in 4% DMSO in phosphate buffer at pH 7.5. The radioactivity still bound to the Aβ 1-40 fibrils at the end of the incubation was detected on FB filters after filtration in a Brandel apparatus using a wash buffer containing 0.1% Triton-X100.

### In Vivo Rat Brain Entry Studies

Brain exposure after i.v administration was determined in rat brains using cassette dosing. Four different compounds were dosed followed by plasma and brain sampling at 2 and 30 minutes after the dosing. 2 to 30 min brain concentration ratios, and percentage of total of injected dose after 2 mins found in brain, were calculated. The compound concentrations were determined by analysis of protein precipitated plasma samples by reversed-phase liquid chromatography coupled to a electrospray tandem mass spectrometer.

### Binding to Amyloid Plaques in Post-Mortem Human AD Brains and Transgenic Mice Brains

Slide-mounted brain sections (10 μm) from APP/PS 1 transgenic mice were collected at the level of the lateral septum (bregma+0.98 mm; see Paxinos and Franklin, 2001). Human cortical sections (7 μm) from two AD patients and 1 control subject were obtained from a Dutch tissue bank.

Sections were preincubated for 30 minutes at room temperature in 50 mM Tris HCl (pH 7.4) in the presence or absence of 1 μM PIB. Sections were transferred to buffer containing tritium-labeled compound (1 nM) with or without PIB (1 μM) and incubated for 30 minutes at room temperature. Incubation was terminated by 3 consecutive 10 minute rinses in buffer (1° C.) followed by a rapid rinse in distilled water (1° C.). Sections were air dried in front of a fan. Dried sections and plastic tritium standards (Amersham microscales-3H) were apposed to phosphoimage plates (Fuji) in a cassette and exposed overnight. The following morning, the image plates were processed with a Fuji phosphoimager (BAS 2500) using BAS Reader software. The resulting image was converted to TIF format using Aida software, optimized with Adobe Photoshop (v 8.0) and quantified using Image-J (NIH). Data were statistically analyzed using Excel.

Binding in APP/PSI Mouse Brain after Compound Administration In-Vivo

Naïve, awake mice were restrained and intravenously infused via the tail vein with either a tritium labeled compound of the present invention, or a tritium labeled reference compound via the tail vein. In one type of experiment, the animals were rapidly anesthetized with isofluorane and decapitated twenty minutes after compound administration (1 mCi). In another type of experiment, mice were given 1 mCi of a compound and were anesthetized and decapitated at a timepoint of 20, 40 or 80 minutes after administration. Brains were removed and frozen with powdered dry ice. Brains were sectioned (10 μm) in the coronal plane at the level of the striatum with a cryostat, thaw-mounted onto superfrost microscope slides and air-dried.

Methods designed to optimize the imaging of bound ligand after in vivo administration were thereafter employed. To selectively reduce unbound radioactivity levels, one-half of the sections were rinsed (3×10 minutes) in cold (1° C.) Tris buffer (50 mM, pH7.4) followed by a rapid rinse in cold (1° C.) deionized water. Sections were then air dried in front of a fan. Rinsed as well as i sections and tritium standards were exposed to phosphoimage plates (Fuji). Phosphoimage plates were processed with a Fujifilm BAS-2500 phosphoimager using BAS Reader software.

### BIOLOGICAL EXAMPLE 1

### Characterization of Specific Binding of Novel 2-heteroaryl Substituted Indole Derivatives to Aβ Amyloid Fibrils In Vitro

Specific binding was determined according to the competion binding assay described herein. The determined IC₅₀'s in the competion binding assays (using [³H]PIB as radioligand) of compounds of the present invention are shown in Table 1.

TABLE 1
IC₅₀'s obtained of exemplified compounds of the present
invention when tested in the competion binding assay.

| NAME | IC50 (nM) |
|---|---|
| methyl 6-(5-hydroxy-1H-indol-2-yl)nicotinate | 140 |
| methyl 6-(5-methoxy-1H-indol-2-yl)nicotinate | 141 |
| 5-(1H-indol-2-yl)pyridine-2-carboxamide | 143 |
| 5-(5-fluoro-1H-indol-2-yl)-N-methylpyridin-2-amine | 152 |
| 5-(5-methoxy-1H-indol-2-yl)-N-methylpyridin-2-amine | 201 |
| 2-[6-(methylamino)pyridin-3-yl]-1H-indol-5-ol | 736 |
| tert-butyl 2-(6-carbamoylpyridin-3-yl)-5-fluoro- | 5370 |
| 1H-indole-1-carboxylate | |

1. A compound according to formula Ia or a pharmaceutically acceptable salt thereof, wherein:
formula Ia corresponds to: 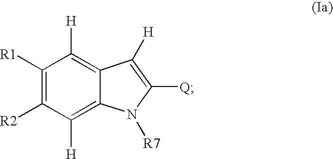
as to R1 and R2:
R1 and R2 are independently selected such that:
R1 is selected from H, halo, methyl, C₁₋₅ fluoroalkyl, C₁₋₃ alkyleneOC₁₋₃ alkyl, C₁₋₃ alkyleneOC₁₋₃ fluoroalkyl, C₁₋₃ alkyleneNH₂, C₁₋₃ alkyleneNHC₁₋₃ alkyl, C₁₋₃ alkyleneN(C₁₋₃ alkyl)₂, C₁₋₃ alkyleneNHC₁₋₃ fluoroalkyl, C₁₋₃ alkyleneN(C₁₋₃ fluoroalkyl)₂, C₁₋₃ alkyleneN(C₁₋₃alkyl)C₁₋₃ fluoroalkyl, hydroxy, methoxy, C₁₋₅ fluoroalkoxy, C₁₋₅ alkylthio, C₁₋₅ fluoroalkylthio, amino, NHC₁₋₃ alkyl, NHC₁₋₃ fluoroalkyl, N(C₁₋₃alkyl)₂, N(C₁₋₃ alkyl)C₁₋₃ fluoroalkyl, NH(CO)C₁₋₃ alkyl, NH(CO)C₁₋₃ fluoroalkyl, NH(CO)C₁₋₃alkoxy, NH(CO)C₁₋₃ fluoroalkoxy, NHSO₂C₁₋₃ alkyl, NHSO₂C₁₋₃ fluoroalkyl, (CO)C₁₋₃ alkyl, (CO)C₁₋₃ fluoroalkyl, (CO)C₁₋₃alkoxy, (CO)C₁₋₃ fluoroalkoxy, (CO)NH₂, (CO)NHC₁₋₃ alkyl, (CO)NHC₁₋₃ fluoroalkyl, (CO)N(C₁₋₃alkyl)₂, (CO)N(C₁₋₃ alkyl)C₁₋₃ fluoroalkyl, (CO)N(C₄₋₆alkylene), (CO)N(C₄₋₆ fluoroalkylene), cyano, SO₂NHC₁₋₃ fluoroalkyl, nitro, and SO₂NH₂; and
R2 is selected from H, halo, methyl, C₁₋₅ fluoroalkyl, C₁₋₃ alkyleneOC₁₋₃ alkyl, C₁₋₃ alkyleneOC₁₋₃ fluoroalkyl, C₁₋₃ alkyleneNH₂, C₁₋₃ alkyleneNHC₁₋₃ alkyl, C₁₋₃ alkyleneN(C₁₋₃ alkyl)₂, C₁₋₃ alkyleneNHC₁₋₃ fluoroalkyl, C₁₋₃ alkyleneN(C₁₋₃ fluoroalkyl)₂, C₁₋₃ alkyleneN(C₁₋₃alkyl)C₁₋₃ fluoroalkyl, hydroxy, methoxy, C₁₋₅ fluoroalkoxy, C₁₋₅ alkylthio, C₁₋₅ fluoroalkylthio, amino, NHC₁₋₃ alkyl, NHC₁₋₃ fluoroalkyl, N(C₁₋₃alkyl)₂, N(C₁₋₃ alkyl)C₁₋₃ fluoroalkyl, NH(CO)C₁₋₃ alkyl, NH(CO)C₁₋₃ fluoroalkyl, NH(CO)C₁₋₃alkoxy, NH(CO)C₁₋₃ fluoroalkoxy, NHSO₂C₁₋₃ alkyl, NHSO₂C₁₋₃ fluoroalkyl, (CO)C₁₋₃ alkyl, (CO)C₁₋₃ fluoroalkyl, (CO)C₁₋₃alkoxy, (CO)C₁₋₃ fluoroalkoxy, (CO)NH₂, (CO)NHC₁₋₃ alkyl, (CO)NHC₁₋₃ fluoroalkyl, (CO)N(C₁₋₃alkyl)₂, (CO)N(C₁₋₃ alkyl)C₁₋₃ fluoroalkyl, (CO)N(C₄₋₆alkylene), (CO)N(C₄₋₆ fluoroalkylene), cyano, SO₂NHC₁₋₃ fluoroalkyl, nitro, and SO₂NH₂; or
R1 and R2 together form: 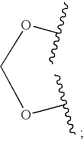
Q is a nitrogen-containing aromatic heterocycle selected from Q2, Q3, Q4, Q5, Q6, Q7, Q8, Q9, and Q10: 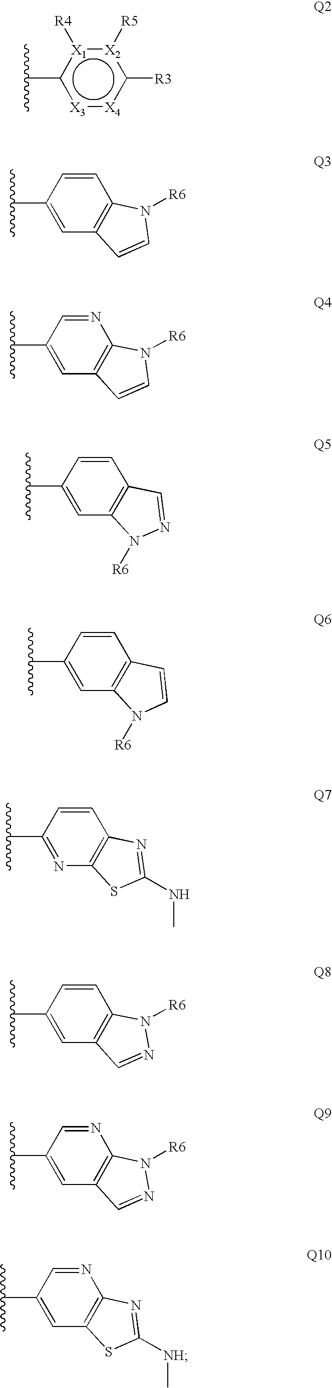
Q2 is a 6-membered aromatic heterocycle containing one or two N atoms;
one or two of X₁, X₂, X₃, and X₄ is/are N, and the remaining are C;
when X₁ is C, the C is optionally substituted with R4;
when X₂ is C, the C is optionally substituted with R5;
R3 is selected from methoxy, C₁₋₄ fluoroalkoxy, amino, NHC₁₋₃ alkyl, NHC₁₋₃ fluoroalkyl, N(C₁₋₃alkyl)₂, N(C₁₋₃alkyl)C₁₋₃ fluoroalkyl, NH(CO)C₁₋₃ alkyl, NH(CO)C₁₋₃ fluoroalkyl, NH(CO)G2, (CO)NH₂, (CO)C₁₋₃ alkoxy, methylthio, C₁₋₆ fluoroalkylthio, SO₂NH₂, N(C₄₋₆ alkylene), and G1: 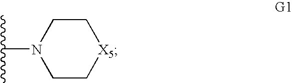
X₅ is selected from O, NH, NC₁₋₃ alkyl, and N(C0)Ot-butyl;
G2 is phenyl optionally substituted with a substituent selected from fluoro and iodo;
R4 is selected from H, fluoro, bromo, and iodo;
R5 is selected from H, fluoro, bromo, and iodo;
R6 is selected from H, methyl, and (CH₂)₀₋₄CH₂F;
R7 is selected from H, methyl, (CO)C₁₋₄alkoxy, and (CH₂)₀₋₄CH₂F;
one or more of the atoms of formula Ia optionally is/are a detectable isotope; and
the compound is not any of the following compounds:

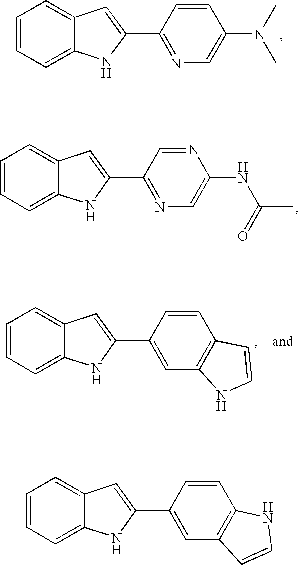

2. A compound or pharmaceutically acceptable salt thereof according to claim 1, wherein:
R1 is an independent substituent such that R1 is selected from H, halo, methyl, C₁₋₅ fluoroalkyl, hydroxy, methoxy, C₁₋₅ fluoroalkoxy, methylthio, C₁₋₅ fluoroalkylthio, amino, NHmethyl, NHC₁₋₃ fluoroalkyl, N(CH₃)CH₃, N(C₁₋₃alkyl)C₁₋₃ fluoroalkyl, NH(CO)C₁₋₃ alkyl, NH(CO)C₁₋₃ fluoroalkyl, NH(CO)C₁₋₃ alkoxy, NH(CO)C₁₋₃ fluoroalkoxy, NHSO₂C₁₋₃ alkyl, NHSO₂C₁₋₃ fluoroalkyl, (CO)C₁₋₃ fluoroalkyl, (CO)C₁₋₃alkoxy, (CO)C₁₋₃ fluoroalkoxy, (CO)NH₂, (CO)NHC₁₋₃ fluoroalkyl, cyano, SO₂NHC₁₋₃ fluoroalkyl, nitro and SO₂NH₂; or
R1 and R2 together form:

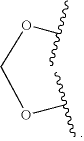

3. A compound or pharmaceutically acceptable salt thereof according to claim 1, wherein R1 is selected from H, fluoro, iodo, methyl, C₁₋₅ fluoroalkyl, hydroxy, methoxy, cyano, C₁₋₅ fluoroalkoxy, methylthio, amino, NHmethyl, NHC₁₋₃ fluoroalkyl, NH(CO)C₁₋₃ alkyl, NH(CO)C₁₋₃ fluoroalkyl, NH(CO)C₁₋₃ fluoroalkoxy, (CO)C₁₋₃alkoxy, and (CO)NH₂.

4. A compound or pharmaceutically acceptable salt thereof according to claim 1, wherein R1 is selected from H, fluoro, hydroxy, and methoxy.

5. A compound or pharmaceutically acceptable salt thereof according to claim 1, wherein R2 is selected from H, fluoro, iodo, C₁₋₅ fluoroalkyl, hydroxy, methoxy, (CO)NH₂, cyano, and methylthio.

6. A compound or pharmaceutically acceptable salt thereof according to claim 1, wherein R2 is selected from H, fluoro, hydroxy, and methoxy.

7. A compound or pharmaceutically acceptable salt thereof according to claim 1, wherein R2 is H.

8. A compound or pharmaceutically acceptable salt thereof according to claim 1, wherein Q is Q2.

9. A compound or pharmaceutically acceptable salt thereof according to claim 1, wherein Q is selected from Q3, Q4, Q5, Q6, Q7, Q8, Q9, and Q10.

10. A compound or pharmaceutically acceptable salt thereof according to claim 1, wherein:
Q2 is pyridine; and
one of X₃ and X₄ is N,
one of X₃ and X₄ is C, and
X₁ and X₂ are C.

11. A compound or pharmaceutically acceptable salt thereof according to claim 1,
Q2 is pyrimidine,
X₂ and X₄ are N, and
X₁ and X₃ are C.

12. A compound or pharmaceutically acceptable salt thereof according to claim 1, wherein:
Q2 is pyrimidine,
X₁ and X₃ are N, and
X₂ and X₄ are C.

13. A compound or pharmaceutically acceptable salt thereof according to claim 1, wherein:
Q2 is pyridazine,
X₃ and X₄ are N, and
X₁ and X₂ are C.

14. A compound or pharmaceutically acceptable salt thereof according to claim 1, wherein:
Q2 is pyrazine; and
as to X₁, X₂, X₃, and X₄:
X₁ and X₄ are N, and X₂ and X₃ are C; or
X₁ and X₄ are C, and X₂ and X₃ are N.

15. A compound or pharmaceutically acceptable salt thereof according to claim 10, wherein:
R3 is selected from methoxy, C₁₋₄ fluoroalkoxy, amino, NHC₁₋₃ alkyl, NHC₁₋₃ fluoroalkyl, N(C₁₋₃alkyl)₂, N(C₁₋₃alkyl)C₁₋₃ fluoroalkyl, NH(CO)C₁₋₃ alkyl, NH(CO)C₁₋₃ fluoroalkyl, (CO)NH₂, (CO)C₁₋₃alkoxy, methylthio, C₁₋₆ fluoroalkylthio, SO₂NH₂, and G1; and
X₅ is selected from O, NH_{S} and Nmethyl.

16. A compound or pharmaceutically acceptable salt thereof according to claim 10, wherein R3 is selected from NHmethyl, (CO)NH₂, and (CO)methoxy.

17. A compound or pharmaceutically acceptable salt thereof according to claim 10, wherein R4 is fluoro.

18. A compound or pharmaceutically acceptable salt thereof according to claim 10, wherein R4 is H.

19. A compound or pharmaceutically acceptable salt thereof according to claim 10, wherein R5 is fluoro.

20. A compound or pharmaceutically acceptable salt thereof according to claim 10, wherein R5 is H.

21. A compound or pharmaceutically acceptable salt thereof according to claim 10, wherein R6 is methyl.

22. A compound or pharmaceutically acceptable salt thereof according to claim 10, wherein R6 is H.

23. A compound or pharmaceutically acceptable salt thereof according to claim 1, wherein R7 is methyl.

24. A compound or pharmaceutically acceptable salt thereof according to claim 1, wherein R7 is H or (CO)C₁₋₄alkoxy.

25. A compound or pharmaceutically acceptable salt thereof according to claim 1, wherein the compound is:

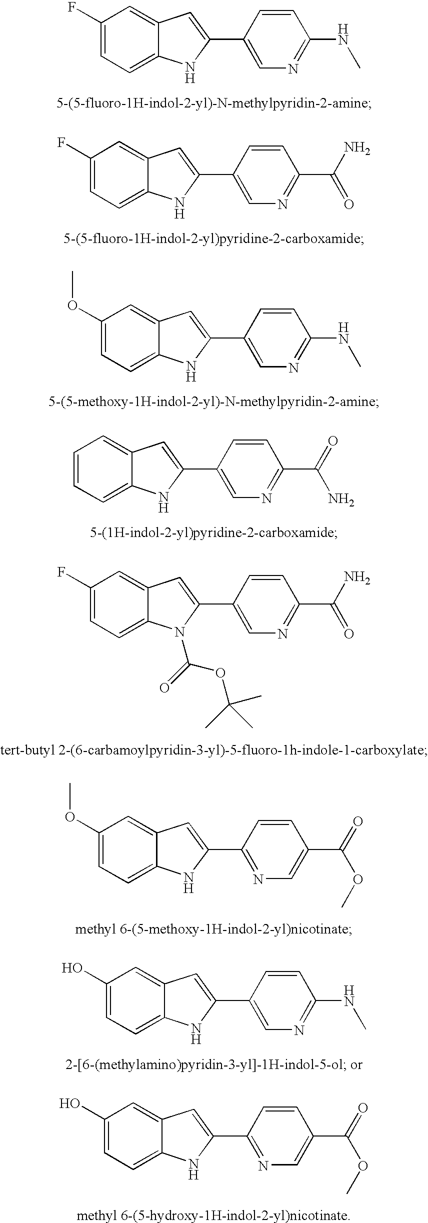

26. A compound or pharmaceutically acceptable salt thereof, wherein:
either:
one to six of the composing atoms is/are ³H,
one to three of the composing atoms is/are ¹³C, or
one of the composing atoms is selected from ¹⁸F, ¹¹C, ⁷⁵Br, ⁷⁶Br, ¹²⁰I, ¹²³I, ¹²⁵I, ¹³¹I, and ¹¹C; and
the compound is selected from:

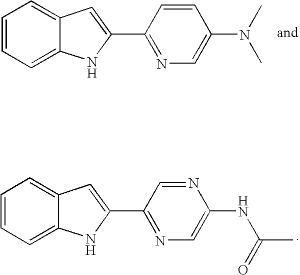

27. A compound or pharmaceutically acceptable salt thereof according to claim 26, wherein one of the composing atoms is ¹¹C.

28. A compound or pharmaceutically acceptable salt thereof according to claim 1, wherein one or more of the atoms of the molecule is/are a detectable isotope.

29. A compound or pharmaceutically acceptable salt thereof according to claim 1, wherein:
one to six of the composing atoms is/are ³H,
one to three of the composing atoms is/are selected from ¹⁹F and ¹¹C, or
one of the composing atoms is selected from ¹⁸F, ¹¹C, ⁷⁵Br, ⁷⁶Br, ¹²¹I, ¹²³I, ¹²⁵I, ¹³¹I, and ¹¹C.

30. A compound or pharmaceutically acceptable salt thereof according to claim 1, wherein:
one to six of the composing atoms is/are ³H,
one to three of the composing atoms is/are ¹⁹F, or
one of the composing atoms is selected from ¹⁸F, ¹¹C, and ¹²³I.

31. A compound or pharmaceutically acceptable salt thereof according to claim 1, wherein:
one to six of the composing atoms is/are ³H,
one to three of the composing atoms is/are ¹⁹F, or
one of the composing atoms is selected from ¹⁸F and ¹¹C.

32. A compound or pharmaceutically acceptable salt thereof according to claim 1, wherein one of the composing atoms is ¹¹C.

33. A compound or pharmaceutically acceptable salt thereof according to claim 1, wherein one of the composing atoms is ¹⁸F.

34. A compound according to formula Ib or a salt thereof, wherein:
formula Ib corresponds to: 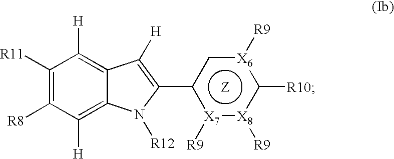
Z is a 6-membered aromatic heterocycle containing one or two N atoms;
one or two of X₆, X₇ and X₈ is/are N, and the remaining are C;
when X₆ is C, the C is optionally substituted with R9;
when X₇ is C, the C is optionally substituted with R9;
when X₈ is C, the C is optionally substituted with R9;
R8 is selected from OSi(G3)₃, OCH₂G4, OG5, H, bromo, fluoro, hydroxy, methoxy, Sn(C₁₋₄alkyl)₃, N(CH₃)₃⁺, IG6⁺, N₂⁺ and nitro;
R9 is selected from H, bromo, fluoro, chloro, iodo, Sn(C₁₋₄ alkyl)₃, N(CH₃)₃⁺, IG6⁺, N₂⁺, and nitro;
R10 is selected from amino, methylamino, dimethylamino, N(CH₃)CHO, N(CH₃)COCH₃, N(CH₃)CO₂-t-butyl, methoxy, hydroxy, (CO)NH₂, O(CH₂)₂₋₄G7 and NH(CH₂)₂₋₄G7;
R11 is selected from OSi(G3)₃, OCH₂G4, OG5, H, bromo, fluoro, hydroxy, methoxy, Sn(C₁₋₄ alkyl)₃, N(CH₃)₃⁺, IG6⁺, N₂⁺ and nitro;
R12 is selected from H, methyl, SO₂N(CH₃)₂, SO₂phenyl, SO₂(p-methyl)phenyl, CO₂CH₂CCl₃, CO₂(CH₂)₂Si(CH₃)₂, CO₂t-butyl, Si(G3)₃, P(═S)phenyl₂, and (CH₂)₂₋₄G7;
G3 is selected from C₁₋₄ alkyl and phenyl;
G4 is selected from 2-(trimethylsilyl)ethoxy, C₁₋₃ alkoxy, 2-(C₁₋₃ alkoxy)ethoxy, C₁₋₃ alkylthio, cyclopropyl, vinyl, phenyl, p-methoxyphenyl, o-nitrophenyl, and 9-anthryl;
G5 is selected from tetrahydropyranyl, 1-ethoxyethyl, phenacyl, 4-bromophenacyl, cyclohexyl, t-butyl, t-butoxycarbonyl, 2,2,2-trichloroethylcarbonyl and triphenylmethyl;
IG6⁺ is a constituent of a iodonium salt, in which the iodo atom is hyper-valent and has a positive formal charge and, in which G6 is phenyl, optionally substituted with one substituent selected from methyl and bromo;
G7 is selected from bromo, iodo, OSO₂CF₃, OSO₂CH₃ and OSO₂-phenyl, wherein:
the phenyl is optionally substituted with methyl or bromo;
with reference to formula Ib, one or both of the following conditions are fulfilled:
(1) R12 is H; and
(2) one or several of R8, R9, R10, and R11 is/are selected from bromo, fluoro, hydroxy, Sn(C₁₋₄ alkyl)₃, N(CH₃)₃⁺, IG6⁺, N₂⁺, nitro, amino, methylamino, NH(CH₂)₂₋₄G7, N(CH₃)CHO, N(CH₃)COCH₃, N(CH₃)CO₂-t-butyl, 0(CH₂)₂₋₄G7, OSi(G3)₃, OCH₂G4, (CH₂)₂₋₄G7; and
the compound is not either of the following compounds:

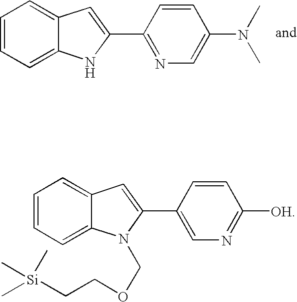

35. A compound or salt thereof according to claim 34, wherein:
R8 is H;
R9 is selected from H, fluoro, and nitro;
R10 is selected from amino, methylamino, dimethylamino, NH(CH₂)₂₋₄G7, N(CH₃)CHO, N(CH₃)COCH₃, N(CH₃)CO₂-t-butyl, (CO)NH₂, and O(CH₂)₂₋₄G7;
R11 is selected from OSi(CH₃)₂C(CH₃)₃, H, fluoro, hydroxy, methoxy, OCH₂G4, Sn(C₁₋₄ alkyl)₃, and N₂⁺; and
R12 is selected from H, SO₂(p-methyl)phenyl, CO₂(CH₂)₂Si(CH₃)₂, CO₂t-butyl, Si(CH₃)₂C(CH₃)₃, and P(═S)phenyl₂.

36. A compound or salt thereof according to claim 34, wherein:
Z is a 6-membered aromatic heterocycle, and
X₆, X₇, and X₈ are C.

37. A compound or salt thereof according to claim 34, wherein:
Z is pyridine,
X₆ and X₇ are C, and
X₈ is N.

38. A compound or salt thereof according to claim 34, wherein:
Z is pyridine,
X₆ and X₈ are C, and
X₇ is N.

39. A compound or salt thereof according to claim 34, wherein:
Z is pyrimidine,
X₆ and X₈ are N, and
X₇ is C.

40. A compound or salt thereof according to claim 34, the compound is:

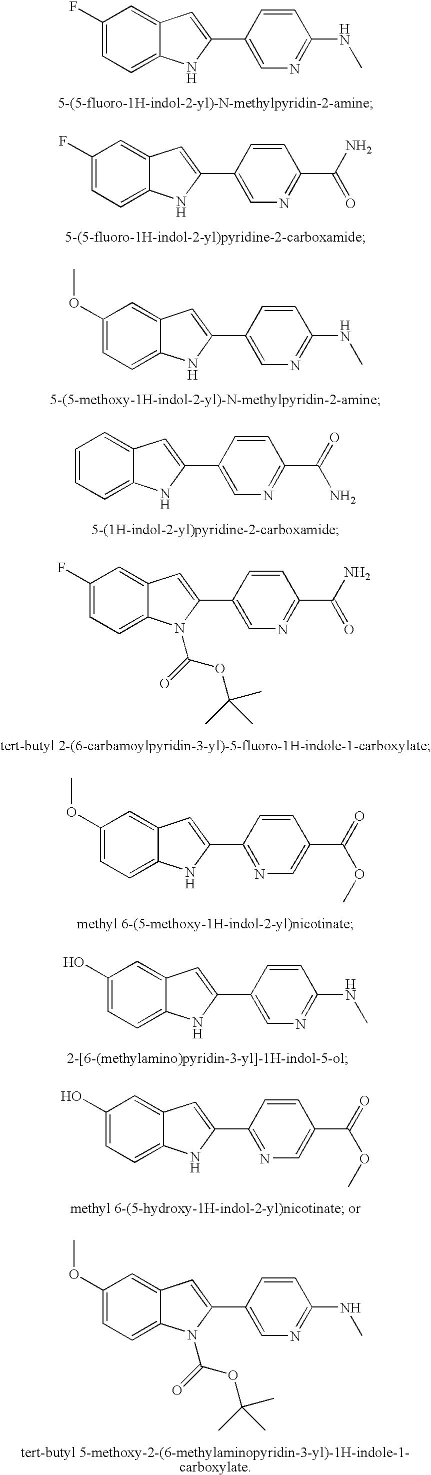

41. A process for making a labeled compound or a salt thereof, wherein:
the process comprises use of a compound of formula (Ib) or a salt thereof as synthetic precursor to prepare the labeled compound or salt thereof;
the labeled compound or salt thereof is a compound or a pharmaceutically acceptable salt thereof according to claim 28; comprising one [¹¹C]methyl group;
formula Ib corresponds to: 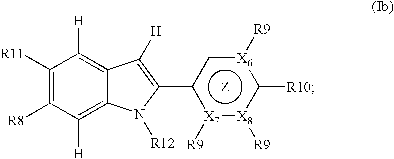
Z is a 6-membered aromatic heterocycle containing one or two N atoms;
one or two of X₆, X₇, and X₈ is/are N and the remaining are C;
when X₆ is C the C is optionally substituted with R9;
when X₇ is C the C is optionally substituted with R9;
when X₈ is C the C is optionally substituted with R9;
R8 is selected from OSi(G3)₃, OCH₂G4, OG5H bromo, fluoro, hydroxy, methoxy, Sn(C₁₋₄ alkyl)₃, N(CH₃)₃, IG6⁺, N₂⁺ and nitro;
R9 is selected from H bromo, fluoro, chloro, iodo, Sn(C₁₋₄ alkyl)₃, N(CH₃)₃⁺, IG6⁺, N₂⁺, and nitro;
R10 is selected from amino, methylamino, dimethylamino, N(CH₃)CHO, N(CH₃)COCH₃, N(CH₃)CO₂-t-butyl, methoxy, hydroxy, (CO)NH₂, O(CH₂)₂₋₄G7, and NH(CH₂)₂₋₄G7;
R11 is selected from OSi(G3)₃OCH₂G4, OG5, H bromo, fluoro, hydroxy, methoxy, Sn(C₁₋₄ alkyl)₃, N(CH₃)₃⁺, IG6⁺, N₂⁺ and nitro;
R12 is selected from H, methyl, SO₂N(CH₃)₂, SO₂phenyl, SO₂(p-methyl)phenyl, CO₂CH₂CCl₃, CO₂(CH₂)₂Si(CH₃)₂, CO₂t-butyl, Si(G3)₃, P(═S)phenyl₂, and (CH₂)₂₋₄G7;
G3 is selected from C₁₋₄ alkyl and phenyl;
G4 is selected from 2-(trimethylsilyl)ethoxy, C₁₋₃ alkoxy, 2-(C₁₋₃ alkoxy)ethoxy, C₁₋₃ alkylthio, cyclopropyl, vinyl, phenyl, p-methoxyphenyl, o-nitrophenyl, and 9-anthryl;
G5 is selected from tetrahydropyranyl, 1-ethoxyethyl, phenacyl, 4-bromophenacyl, cyclohexyl, t-butyl, t-butoxycarbonyl, 2,2,2-trichloroethylcarbonyl, and triphenylmethyl;
IG6⁺ is a constituent of a iodonium salt, in which the iodo atom is hyper-valent and has a positive formal charge and, in which G6 is phenyl, optionally substituted with one substituent selected from methyl and bromo;
G7 is selected from bromo iodo OSO₂CF₃, OSO₂CH₃, and OSO₂phenyl, wherein:
the phenyl is optionally substituted with methyl or bromo;
with reference to formula Ib, one or both of the following conditions is/are fulfilled:
(1) R12 is H; and
(2) one or several of R8, R9, R10, and R11 is/are selected from bromo, fluoro, hydroxy, Sn(C₁₋₄ alkyl)₃, N(CH₃)₃⁺, IG6⁺, N₂⁺, nitro, amino, methylamino, NH(CH₂)₂₋₄G7, N(CH₃)CHO, N(CH₃)COCH₃, N(CH₃)CO₂-t-butyl, O(CH₂)₂₋₄G7, OSi(G3)₃, OCH₂G4, (CH₂)₂₋₄G7; and
the compound is not either of the following compounds:

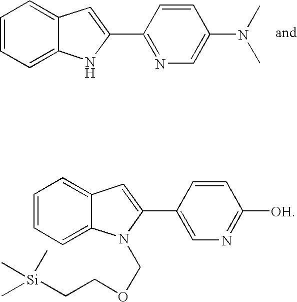

42. A process for making a labeled compound or a salt thereof, wherein:
the process comprises use of a compound of formula (Ib) or a salt thereof as synthetic precursor to prepare the labeled compound or salt thereof;
the labeled compound or salt thereof is a compound or pharmaceutically acceptable salt thereof according to claim 29; comprising one ¹⁸F atom;
formula Ib corresponds to: 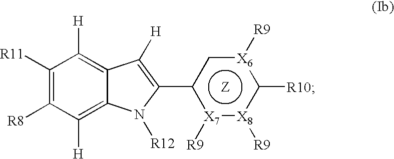
Z is a 6-membered aromatic heterocycle containing one or two N atoms;
one or two of X₆, X₇, and X₈ is/are N, and the remaining are C;
when X₆ is C, the C is optionally substituted with R9;
when X₇ is C, the C is optionally substituted with R9;
when X₈ is C, the C is optionally substituted with R9;
R8 is selected from OSi(G3)₃, OCH₂G4, OG5, H, bromo, fluoro, hydroxy, methoxy, Sn(C₁₋₄ alkyl)₃, N(CH₃)₃⁺, IG6⁺, N₂⁺, and nitro;
R9 is selected from H, bromo, fluoro, chloro, iodo, Sn(C₁₋₄ alkyl)₃, N(CH₃)₃⁺, IG6⁺, N₂⁺, and nitro;
R10 is selected from amino, methylamino, dimethylamino, N(CH₃)CHO, N(CH₃)COCH₃, N(CH₃)CO₂-t-butyl, methoxy, hydroxy, (CO)NH₂, O(CH₂)₂₋₄G7, and NH(CH₂)₂₋₄G7;
R11 is selected from OSi(G3)₃, OCH₂G4, OG5, H, bromo, fluoro, hydroxy, methoxy, Sn(C₁₋₄ alkyl)₃, N(CH₃)₃⁺, IG6⁺, N₂⁺, and nitro;
R12 is selected from H, methyl, SO₂N(CH₃)₂, SO₂phenyl, SO₂(p-methyl)phenyl, CO₂CH₂CCl₃, CO₂(CH₂)₂Si(CH₃)₂, CO₂t-butyl, Si(G3)₃, P(═S)phenyl₂, and (CH₂)₂₋₄G7;
G3 is selected from C₁₋₄ alkyl and phenyl;
G4 is selected from 2-(trimethylsilyl)ethoxy, C₁₋₃ alkoxy, 2-(C₁₋₃ alkoxy)ethoxy, C₁₋₃ alkylthio, cyclopropyl, vinyl, phenyl, p-methoxyphenyl, o-nitrophenyl, and 9-anthryl;
G5 is selected from tetrahydropyranyl, 1-ethoxyethyl, phenacyl, 4-bromophenacyl, cyclohexyl, t-butyl, t-butoxycarbonyl, 2,2,2-trichloroethylcarbonyl, and triphenylmethyl;
IG6⁺ is a constituent of a iodonium salt, in which the iodo atom is hyper-valent and has a positive formal charge and, in which G6 is phenyl, optionally substituted with one substituent selected from methyl and bromo;
G7 is selected from bromo iodo OSO₂CF₃, OSO₂CH₃, and OSO₂phenyl, wherein:
the phenyl is optionally substituted with methyl or bromo;
with reference to formula Ib, one or both of the following conditions is/are fulfilled:
(1) R12 is H; and
(2) one or several of R8, R9, R10, and R11 is/are selected from bromo, fluoro, hydroxy. Sn(C₁₋₄ alkyl)₃, N(CH₃)₃⁺, IG6⁺, N₂⁺, nitro, amino methylamino NH(CH₂)₂₋₄G7, N(CH₃)CHO, N(CH₃)COCH₃, N(CH₃)CO₂-t-butyl, O(CH₂)₂₋₄G7, OSi(G3)₃, OCH₂G4, (CH₂)₂₋₄G7; and
the compound is not either of the following compounds:

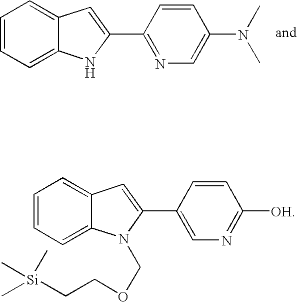

43. A process for making a labeled compound or a salt thereof, wherein:
the process comprises use of a compound of formula (Ib) or a salt thereof as synthetic precursor to prepare the labeled compound or salt thereof;
the labeled compound or salt thereof is a compound or a pharmaceutically acceptable salt thereof according to claim 27, comprising one atom selected from ¹²⁰I, ¹²³I, ¹²⁵I, and ¹³¹I;
formula Ib corresponds to: 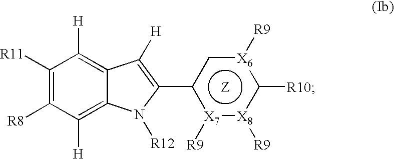
Z is a 6-membered aromatic heterocycle containing one or two N atoms;
one or two of X₆, X₇, and X₈ is/are N and the remaining are C;
when X₆ is C, the C is optionally substituted with R9;
when X₇ is C, the C is optionally substituted with R9;
when X₈ is C, the C is optionally substituted with R9;
R8 is selected from OSi(G3)₃, OCH₂G4, OG5, H, bromo, fluoro, hydroxy, methoxy, Sn(C₁₋₄ alkyl)₃, N(CH₃)₃, IG6⁺, N₂⁺, and nitro;
R9 is selected from H, bromo, fluoro, chloro, iodo Sn(C₁₋₄ alkyl)₃, N(CH₃)₃⁺, IG6⁺, N₂⁺, and nitro;
R10 is selected from amino, methylamino, dimethylamino, N(CH₃)CHO, N(CH₃)COCH₃, N(CH₃)CO₂-t-butyl, methoxy, hydroxy, (CO)NH₂, O(CH₂)₂₋₄G7, and NH(CH₂)₂₋₄G7;
R11 is selected from OSi(G3)₃, OCH₂G4, OG5, H, bromo, fluoro, hydroxy, methoxy, Sn(C₁₋₄ alkyl)₃, N(CH₃)₃⁺, IG6⁺, N₂⁺, and nitro;
R12 is selected from H, methyl, SO₂N(CH₃)₂, SO₂phenyl, SO₂(p-methyl)phenyl, CO₂CH₂CCl₃, CO₂(CH₂)₂Si(CH₃)₂, C)₂t-butyl, Si(G3)₃, P(═S)phenyl₂, and (CH₂)₂₋₄G7;
G3 is selected from C₁₋₄ alkyl and phenyl;
G4 is selected from 2-(trimethylsilyl)ethoxy, C₁₋₃ alkoxy, 2-(C₁₋₃ alkoxy)ethoxy, C₁₋₃ alkylthio, cyclopropyl, vinyl, phenyl, p-methoxyphenyl, o-nitrophenyl, and 9-anthryl;
G5 is selected from tetrahydropyranyl, 1-ethoxyethyl, phenacyl, 4-bromophenacyl, cyclohexyl, t-butyl, t-butoxycarbonyl, 2,2,2-trichloroethylcarbonyl, and triphenylmethyl;
IG6⁺ is a constituent of a iodonium salt, in which the iodo atom is hyper-valent and has a positive formal charge and, in which G6 is phenyl, optionally substituted with one substituent selected from methyl and bromo;
G7 is selected from bromo iodo OSO₂CF₃, OSO₂CH₃, and OSO₂phenyl, wherein:
the phenyl is optionally substituted with methyl or bromo;
with reference to formula Ib, one or both of the following conditions is/are fulfilled:
(1) R12 is H; and
(2) one or several of R8, R9, R10, and R11 is/are selected from bromo, fluoro, hydroxy, Sn(C₁₋₄ alkyl)₃, N(CH₃)₃⁺, IG6⁺, N₂⁺, nitro, amino, methylamino, NH(CH₂)₂₋₄G7, N(CH₃)CHO, N(CH₃)COCH₃, N(CH₃)CO₂-t-butyl, O(CH₂)₂₋₄G7, OSi(G3)₃, OCH₂G4, (CH₂)₂₋₄G7; and
the compound is not either of the following compounds:

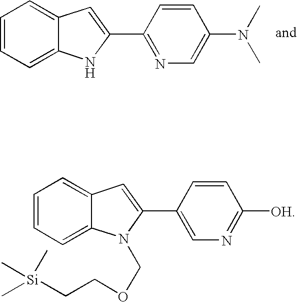

44. A pharmaceutical composition comprising a compound or pharmaceutically acceptable salt thereof according to claim 1, together with a pharmaceutically acceptable carrier.

45. A pharmaceutical composition for in vivo imaging of amyloid deposits, wherein the composition comprises:
a radio-labeled compound or pharmaceutically acceptable salt thereof according to claim 1, and
a pharmaceutically acceptable carrier.

46. An in vivo method for measuring amyloid deposits in a subject, wherein the method comprises:
administering a pharmaceutical composition according to claim 45, and
detecting the binding of the compound or pharmaceutically acceptable salt thereof to an amyloid deposit in the subject.

47. The method according to claim 46, wherein the detection is carried out by gamma imaging, magnetic resonance imaging, or magnetic resonance spectroscopy.

48. The method according to claim 46, wherein the subject is suspected of having a disease or syndrome selected from the group consisting of Alzheimer's Disease, familial Alzheimer's Disease, Down's Syndrome, and homozygotes for the apolipoprotein E4 allele.

49-50. (canceled)

51. A method of prevention and/or treatment of Alzheimer's Disease, familial Alzheimer's Disease, Down's Syndrome, and homozygotes for the apolipoprotein E4 allele in a mammal in need of such prevention and/or treatment, wherein the method comprises administering to the mammal a therapeutically effective amount of a compound or pharmaceutically acceptable salt thereof according to claim 1.

52. A method according to claim 51, wherein the mammal is a human.
